(19) United States
(12) Patent Application Publication
(10) Pub. No.: US 2025/0073003 A1
(43) Pub. Date: Mar. 6, 2025

(54) DIGITAL PATIENT SCANNING SYSTEMS AND METHODS

(71) Applicant: ALIGN TECHNOLOGY, INC., San Jose, CA (US)

(72) Inventors: Edi FRIDMAN, Rishon Le Zion (IL); Gilad ELBAZ, Tel Aviv (IL); Moshe ALBOHER, Kadima-Tsoran (IL); Efrat LABZOVSKY, Tel Aviv (IL); Tal VERKER, Ofra (IL)

(73) Assignee: ALIGN TECHNOLOGY, INC., San Jose, CA (US)

(21) Appl. No.: 18/820,622

(22) Filed: Aug. 30, 2024

Related U.S. Application Data
(60) Provisional application No. 63/579,910, filed on Aug. 31, 2023.

(51) Int. Cl. A61C 9/00; G06T 17/00; G16H 30/20

(52) U.S. Cl. CPC A61C 9/0053; G06T 17/00; G16H 30/20; G06T 2210/41

(57) ABSTRACT

A dental scanning system may include an intraoral scanner and an extraoral imaging device including a color imaging camera couplable in electronic communication with the intraoral scanner. The system may include non-transitory computer readable medium with instructions that, when executed by a processor, cause the system to carry out a method. The method may include capturing 2D image data of a patient's face and teeth with the extraoral imaging device, capturing intraoral 3D image data of a dentition of the patient with the intraoral scanner, generating a 3D model of the patient's teeth based on the intraoral 3D image data, and associating the 2D image data of the patient's face with the 3D model.

### RELATED APPLICATIONS

This application claims the benefit under 35 U.S.C. § 119 (e) of U.S. Provisional Patent Application No. 63/579,910, filed Aug. 31, 2023, and titled “DIGITAL PATIENT SYSTEM,” which is incorporated, in its entirety, by this reference.

### BACKGROUND

Dental patient data, such as intraoral scans and images of the patient's teeth and face are gathered from many disparate systems in many different ways. The gathering and generation of the data is cumbersome as a dental professions manages using different devices, systems, and their differing software to capture the information. Then the dental professional uses a hodgepodge of methods to send the data for use in treatment planning.

On the treatment planning end, the treatment planning systems then attempt to work with different data types, formats, and sizes to plan the dental treatment. These system are less than desirable.

### SUMMARY

As will be described in greater detail below, the present disclosure describes various systems and methods for using intraoral scanners and extraoral imaging systems to more accurately image and 3D scan the patient's face and dentition and packager and provide the data in a way that is easily used by a treatment planning system to plan orthodontic and prosthodontic treatments.

In addition, the systems and methods described herein may improve the functioning of a computing device by reducing computing resources and overhead for acquiring and processing images and models of the patient's face and dentition and for treatment planning, thereby improving processing efficiency of the computing device over conventional approaches. These systems and methods may also improve the field of dental treatment by analyzing data to efficiently correct defects in patient's teeth.

### INCORPORATION BY REFERENCE

All patents, applications, and publications referred to and identified herein are hereby incorporated by reference in their entirety and shall be considered fully incorporated by reference even though referred to elsewhere in the application.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1 shows an example patient digitizing system, in accordance with some embodiments.

FIG. 2A shows an extraoral imaging device, in accordance with some embodiments.

FIG. 2B shows an extraoral imaging device, in accordance with some embodiments.

FIG. 3 shows a method using a patient digitizing system, in accordance with some embodiments.

FIG. 4 shows aspects of an intraoral imaging process, in accordance with some embodiments.

FIG. 5 shows aspects of an extraoral imaging process, in accordance with some embodiments.

FIG. 6A shows aspects an extraoral imaging process, in accordance with some embodiments.

FIG. 6B shows examples of extraoral images, in accordance with some embodiments.

FIG. 7A depicts an extraoral imaging sleeve for an intraoral scanner, in accordance with some embodiments.

FIG. 7B depicts the extraoral imaging sleeve on an intraoral scanner, in accordance with some embodiments.

FIG. 8 depicts an intraoral scanner with an extraoral imaging device, in accordance with some embodiments.

FIG. 9A depicts an intraoral scanner with a diagnostic imaging attachment, in accordance with some embodiments.

FIG. 9B depicts an intraoral scanner with a diagnostic imaging attachment, in accordance with some embodiments.

FIG. 9C shows a method using a patient digitizing system, in accordance with some embodiments.

FIG. 9D depicts an orthodontic treatment system showing aspects of a 2D image capture process, in accordance with some embodiments.

FIG. 9E depicts an orthodontic treatment system showing aspects of a 3D scanning process, in accordance with some embodiments.

FIG. 9F depicts an orthodontic treatment system showing aspects of a diagnostic process, in accordance with some embodiments.

FIG. 10A depicts an intraoral scanner with an air attachment, in accordance with some embodiments.

FIG. 10B depicts an orthodontic treatment system showing aspects of using an air attachment, in accordance with some embodiments

FIG. 11 shows a block diagram of an example computing system capable of implementing one or more embodiments described and/or illustrated herein, in accordance with some embodiments.

FIG. 12 shows a block diagram of an example computing network capable of implementing one or more of the embodiments described and/or illustrated herein, in accordance with some embodiments.

FIG. 13 illustrates an exemplary tooth repositioning appliance or aligner that can be worn by a patient in order to achieve an incremental repositioning of individual teeth in the jaw, in accordance with some embodiments.

FIG. 14 illustrates a tooth repositioning system, in accordance with some embodiments.

FIG. 15 shows a method of orthodontic treatment using a plurality of appliances, in accordance with embodiments;

FIG. 16 shows a method for digitally planning an orthodontic treatment, in accordance with embodiments; and

FIG. 17 shows a simplified block diagram of a data processing system, in accordance with embodiments.

### DETAILED DESCRIPTION

The following detailed description provides a better understanding of the features and advantages of the improvements described in the present disclosure in accordance with the embodiments disclosed herein. Although the detailed description includes many specific embodiments, these are provided by way of example only and should not be construed as limiting the scope of the inventions disclosed herein.

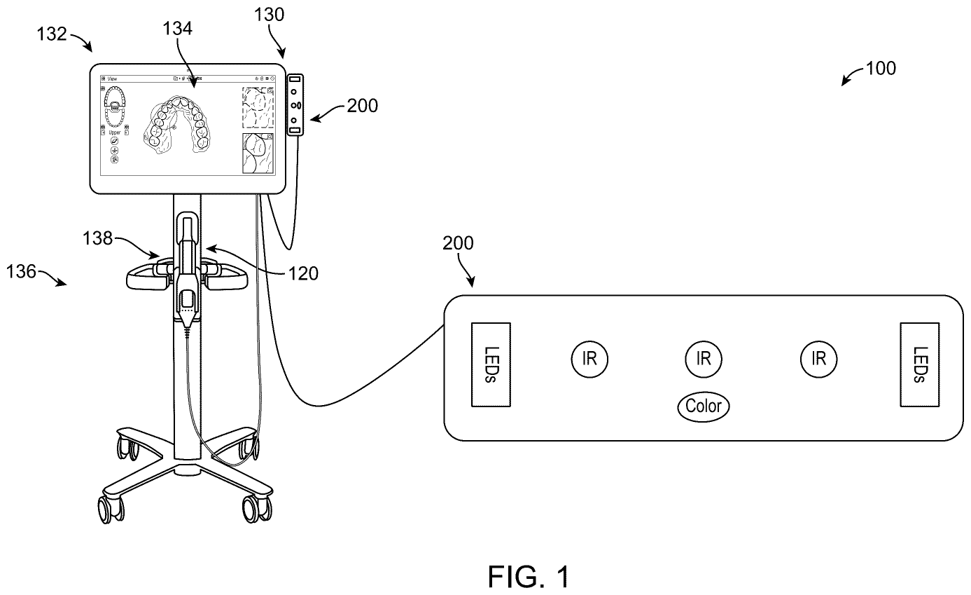

Described herein are patient digitizing systems to generate 2D and 3D data and models of external and internal and subsurface features of a patient's head, including their face, and mouth. The systems may include intraoral scanners for generating three-dimensional (3D) models of a subject's intraoral region (e.g., tooth or teeth, gums, jaw, etc.) which may include surface models of the head, including the face, teeth, and gums, and subsurface features of the teeth, jaws, bones, etc. For example, FIG. 1 a patient digitizing system 100 may include an intraoral imaging device, such as an intraoral scanner 120, that may be configured or adapted as described herein to generate 3D models having both surface and subsurface features. An exemplary intraoral scanner may be hand-held by an operator and moved over a subject's tooth or teeth to scan both surface and internal structures. The wand may include one or more sensors, such as cameras such as CMOS, CCDs, detectors, etc. and one or more light sources. The light sources may be configured to emit light in a first spectral range for detection of surface features, such as visible light or non-visible light and may be monochromatic or polychromatic, a second color light source, such as a white light source that emits light between 400-700 nm or approximately 400-600 nm, and a third light source configured to emit light in a second spectral range for detection of internal features within the tooth, such as by trans-illumination, small-angle penetration imaging, laser florescence, etc., which may generically be referred to as penetration imaging, such as in the near-IR. The light source may be a single light source that emits in the various spectral ranges discussed herein. The light source may include filters to selectively restrict or permit one of more of the spectral ranges discussed herein. The light source may be any appropriate light source, including LED, fiber optic, fluorescent, etc. The probe may include one or more controls (buttons, switching, dials, touchscreens, etc.) to aid in control (e.g., turning the wand on/of, etc.). In some embodiments, alternatively or additionally, one or more controls, not shown, may be present on other parts of the intraoral scanner, such as a foot petal, keyboard, console, touchscreen, etc.

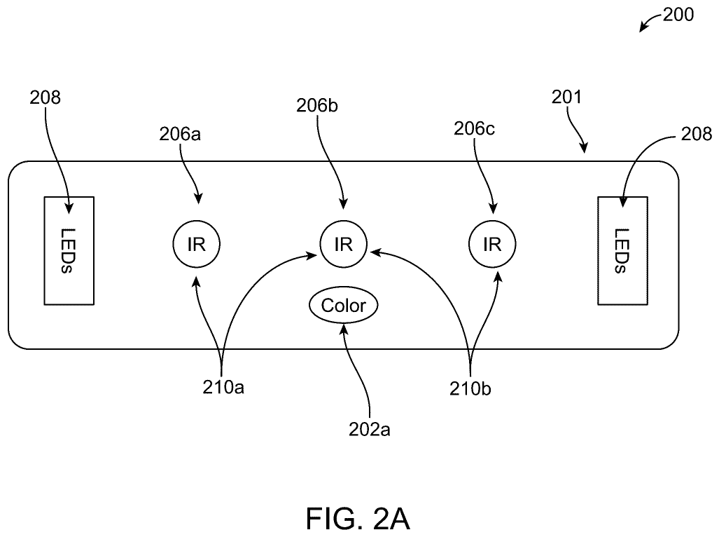

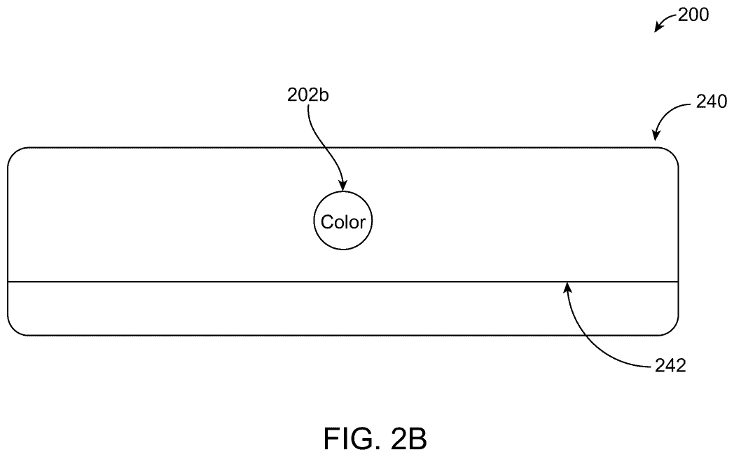

The patient digitizing system 100 may also include an extraoral imaging device 200, that may be configured or adapted as described herein to generate 2D and 3D image data of the surface features of the patient for use in generating a color 3D model of the patient's face and oral cavity and for dental treatment planning, such as orthodontic and prosthodontic treatment planning. An exemplary extraoral scanner 200 may be hand-held by an operator or coupled to or incorporated into the display 132. The hand-held extraoral scanner 200 may be moved about the subject's face and mouth captured 2D and 3D image data of the patient's head and mouth, as discussed in more detail with respect to FIG. 2.

The patient digitizing system 100 may also include a control unit 130. The control unit may also be referend to as a base unit and may control the intraoral imaging device 120 and the extraoral imaging device 200. The control unit 103 may include a monitor 132 for displaying the 2D and 3D data captured by the intraoral imaging device 120 and the extraoral imaging device 200. In some embodiments, the monitor may also include a user interface 134 displayed thereon for guiding the capture of the 2D and 3D data, as described herein.

The patient digitizing system 100 may also include one or more processors, including linked processors or remote processors, for both controlling the intraoral scanner 103 operation, including coordinating the scanning and in reviewing and processing the scanning and generation of the 3D model including surface and internal features. The one or more processors may include or may be coupled with a memory for storing scanned data, such as surface data and subsurface data. Communications circuitry, including wireless or wired communications circuitry may also be included for communicating with components of the system, including the intraoral scanner 103, or external components, including external processors. For example the system may be configured to send and receive scans or 3D models. One or more additional outputs may also be included for outputting or presenting information, including display screens, printers, etc. As mentioned, inputs such as buttons and touchscreens may be included and the apparatus may allow or request user input for controlling scanning and other operations.

Any of the apparatuses and methods described herein may be used to generate 2D and 3D image data and color 3D models for use in dental procedures and treatment planning. Thus, any of the apparatuses described herein may be configured to perform 2D and 3D scans that may be used to determine the shape and color of the anatomy of the patient. Also described herein are methods for using the 2D and 3D image data in smart mirror and other applications.

Moving on to FIGS. 2A and 2B, the extraoral imaging device 200 is shown in greater detail. The extraoral imaging device 200 may include a primary imaging unit 201 and a secondary imaging unity 240. The primary imaging unit 201 of the extraoral imaging device 200 may include multiple infra-red (IR) cameras 206 to capture the 3D data of the patient's face, one or more color cameras 202 to capture high quality color photos of patient's face, and one or more light sources 200 to illuminate the subject.

The primary imaging unit 201 of the extraoral imaging device 200 may include at least two IR cameras 206 that are sensitive to infrared light. The two cameras may work together to generate point clouds for the patient's face and head. The cameras may use structured light, time of flight, or other 3D imaging techniques to generate point clouds that represent the 3D location of the surfaces of the patient's face and head.

Time of flight 3D imaging is a technique used for range imaging to measure the distance between the sensor (or camera) and the subject for every point of the image, to generate a 3D map or depth map of the scene, such as in the form of a point cloud. Infrared light is often used for this purpose because it can provide good range information regardless of visible light conditions and is invisible to the human eye. The “time of flight” is the time taken by the light to travel from the sensor to the object and back.

Time of flight 3D imaging may include modulation ToF methods such as pulsed-modulation or continuous wave modulation time of flight. Pulsed-modulation measures the time-of-flight directly. This generally allows longer distance measurements. Light is emitted in a very short pulse with fast rise- and fall-times and with high optical power-lasers or laser diodes. The departure time of the emitted light and arrival time of the reflected light is measured very precisely.

With continuous wave modulation the phase difference between the sent and received signals is measured. Different shapes of signals are possible, e.g., sinusoidal, square waves, etc. Cross-correlation between the received and sent signals allows phase estimation which is directly related to distance based on a known modulation frequency, typically between 10 MHz and 100 MHZ.

Structured Light 3D imaging is a method to capture 3D information about an object or scene by projecting a known pattern (often lines or grids, sometimes more complex patterns) onto the object or scene, then observing the deformation of the pattern from a different viewpoint.

A light projector casts a structured pattern (a series of lines or a grid, for example) onto the object. This is usually done with infrared light or visible light. The pattern is known and its structure is known. One or more cameras, positioned at known angles relative to the projector, capture images of the pattern as it falls on the object. Because the shape of the object affects how the pattern deforms, these images contain information about the shape of the object.

The system computes the disparity between the known, original pattern and the captured, deformed pattern. This disparity depends on the 3D shape of the object. Using triangulation methods, the system calculates the distance from the camera to each point in the scene. This forms a depth map or a 3D point cloud of the object, with each point having a specific coordinate in the 3D space.

The point cloud data is then processed, usually with specialized software, to create a digital 3D model of the object. Depending on the sophistication of the system, this model could be a simple geometric representation, or it could be a detailed model that includes texture and color information.

The primary imaging unit 201 of the extraoral imaging device 200 may include multiple imaging devices, which may include both a light projector and light sensors, such as a CMOS imaging sensor, to capture the 3D data. The imaging devices 206a, 206b, and 206c, may capture 3D image data simultaneously, such as within less than 60 ms, less than 30 ms, or less than 10 ms. Capturing data simultaneously allows the point clouds generated from each of the cameras to be combined without computationally intensive registration methods that attempt to align two point clouds or other 3D data, such as a 3D model, together into one model or set of 3D data. Instead, because the data is captured in close time proximity and movement of the imaging device is very small, the 3D data from each simultaneously captured image may simply be combined based on the know geometric arrangement of the imaging devices 206a, 206b, and 206c.

In some embodiments, a stereoscopic pair of imagers such as stereoscopic pairs 210a, 210b may be used to generate point clouds. A stereoscopic pair of imagers may include a pair imaging devices in a known spatial relationship that simultaneously image an object from different angles. For example, stereoscopic pair 210a may include IR imager 206a and 206b while stereoscopic pair may include IR imagers 206b and 206c. In some embodiments, IR imagers 206a and 206c may comprise a third stereoscopic pair. The images from a stereoscopic pair may be used to generate a point cloud representing the 3D location of the surfaces of the objects in the field of view of each stereoscopic pair. Stereoscopic imaging may use photogrammetry or other 3D imaging processes to generate the point clouds.

The IR imager may include an IR light sensitive image sensor optically coupled to a lens such that the IR imager has a focal length and field of view to capture the full face from a distance of between 50 cm and 100 cm, preferably as close as 75 cm.

The IR imagers 206 may be arranged in a straight line or may be staggered such that each a line that passes though the centers of IR imager 206a and 206b interests with a line that passes through the centers of IR imager 206b and 206c. The distance between IR imager pairs may be the same, such as shown in FIG. 2A, or they may be different. For example, the distance between imager 206a and 206b may be different that the distance between imagers 206b and 206c.

Although only three IR imagers 206 are depicted in FIG. 2A, the extraoral imaging device 200 may include four IR imagers, which may be arranged in a square, rectangular, rhombus, or diamond arrangement with each imager located at a vertex or a center of a side of the shape. In some embodiments, more than four IR imagers may be used. For example, five imagers may be used. The five imagers may be arranged in a square, rectangular, rhombus, or diamond arrangement with four imagers located at a vertex or a center of a side of the shape and one located in the middle.

The color imager 202 may be a 2D imaging device, such as a color CMOS sensor optically coupled to a lens such that the color imager has a focal length and field of view to capture the full face from a distance of between 50 cm and 100 cm, preferably as close as 75 cm.

The primary imaging unit 201 of the extraoral imaging device 200 may include one or more light sources 208. The light sources 208 may be configured to illuminate the face of the patient with at least twice the ambient illumination at a distance of at least 100 cm. For example, at least 1000 lux. In some embodiments, the light sources may illuminate the face with at least 1400 lux or at least 3000 lux. In some embodiments, the primary imaging unit 201 of the extraoral imaging device 200 may measure or the ambient light on the patient's face and adjust the light source to output at least twice the ambient light lux.

Although the primary imaging unit 201 of the extraoral imaging device 200 is depicted as having two light sources, the primary imaging unit 201 of the extraoral imaging device 200 may include a single light source, or more than two light sources. In some embodiments, the light source may be a light ring. For example, the light source may include a plurality of LEDs arranged in an annular shape behind a diffuser.

The primary imaging unit 201 of the extraoral imaging device 200 may include an energy source, such as batteries. In some embodiments, the primary imaging unit 201 of the extraoral imaging device 200 may receive electrical energy from the control unit 130. For example, the primary imaging unit 201 of the extraoral imaging device 200 may be electrically coupled or wired to the control unit 130.

The dental imaging system 100 may be incorporated into a portable systems, such as a scanning cart 136 having wheels. The control unit 130 maybe incorporated into the cart. For example, the control unit 130 may be mechanically coupled to the cart 136. The intraoral scanner 130 and the extraoral imaging device 200 may also be mechanically coupled to the cart 136, such as via cradles shaped to receive and hold the intraoral scanner 136 and the extra oral imaging device 200. For example, a cradle 138 may hold the intraoral scanner and second cradle may hold the extraoral imaging device 200. In some embodiments, the extraoral imaging device may be held by a cradle attached to the display 132.

The primary imaging unit 201 of the extraoral imaging device 200 may be connected to the controller 130 in electronic communication, such as via USB interface, WiFi, Bluetooth, ethernet, or other data connection.

The extraoral imaging device 200 may include a secondary imaging device 240. The secondary imaging device 240 may include a color imager 202b. The color imager 202 may be a 2D imaging device, such as a color CMOS sensor optically coupled to a lens such that the color imager has a focal length and field of view to capture the full jaw or arch of the patient of between 15 cm and 30 cm, preferably as close as 20 cm.

The secondary imaging unit 240 of the extraoral imaging device 200 may include one or more light sources. The light sources may be configured to illuminate the face of the patient with at least twice the ambient illumination at a distance of at least 30 cm. For example, at least 1000 lux. In some embodiments, the light sources may illuminate the face with at least 1400 lux or at least 3000 lux. In some embodiments, the secondary imaging unit of the extraoral imaging device may measure or the ambient light on the patient's face and adjust the light source to output at least twice the ambient light lux.

The secondary imaging unit 201 of the extraoral imaging device 200 is may include one or more light sources. In some embodiments, the light source may be a light ring. For example, the light source may include a plurality of LEDs arranged in an annular shape behind a diffuser.

The secondary imaging unit 240 of the extraoral imaging device 200 may include an energy source, such as batteries. In some embodiments, the secondary imaging unit 240 of the extraoral imaging device 200 may receive electrical energy from the control unit 130. For example, the secondary imaging unit 240 of the extraoral imaging device 200 may be electrically coupled or wired to the control unit 130 or the primary imaging unit.

The secondary imaging unit 240 of the extraoral imaging device 200 may be connected to the controller 130 and/or the primary imaging unit 201 in electronic communication, such as via USB interface, WiFi, Bluetooth, ethernet, or other data connection.

The primary imaging unit 201 of the extraoral imaging device 200 may include an energy source, such as batteries. In some embodiments, the primary imaging unit 201 of the extraoral imaging device 200 may receive electrical energy from the control unit 130. For example, the primary imaging unit 201 of the extraoral imaging device 200 may be electrically coupled or wired to the control unit 130.

The secondary imaging unit may be releasably coupled to the primary imaging unit. For example, the extraoral imaging unit 200 depicts a seam 242 that shown where the primary imaging unit 201 separates from the secondary imaging unit 240.

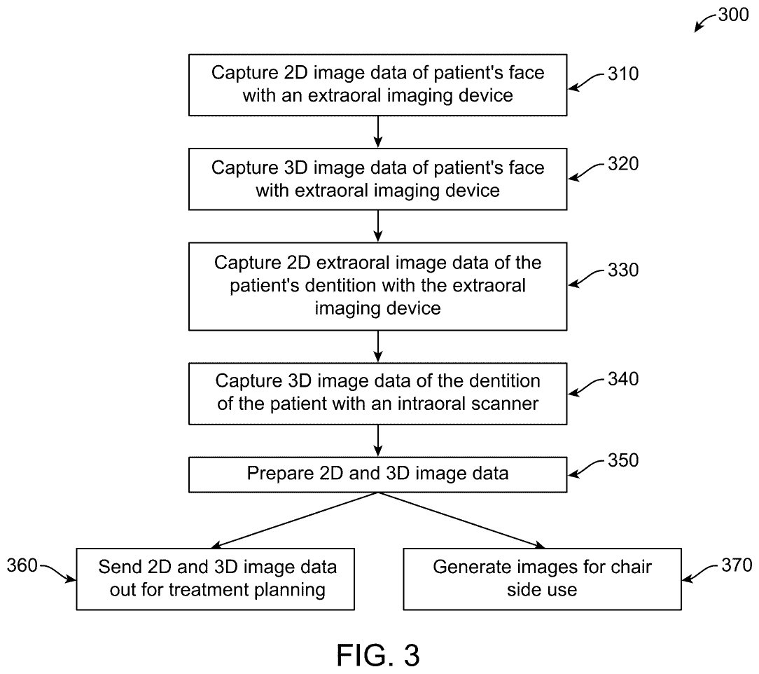

FIG. 3 shows a method 300 of using a patient digitizing system. The method may include capturing 2D image data of the patient's face with an extraoral imaging device at block 310, capturing 3D image data of the patient's face with an extraoral imaging device at block 320, capturing 2D extraoral image data of the patient's dentition with the extraoral imaging device at block 330, capturing 3D image data of the dentition of the patient with an intraoral imaging device, such as an intraoral scanner at block 340, preparing the 2D and 3D image data at block 350, generating images for chair side use at block 360, and outputting the 2D and 3D image data for treatment planning at block 370.

At block 310 the control unit 130 may activate the extraoral imaging device 200 for capturing 2D image data of the patient's face. The control unit 130 may display a prompt to the user to guide the user in capturing the 2D image data of the patient's face. For example, the display may include a user interface to receive input from the user indicating that the user is ready to capture the 2D image data of the patient's face.

As depicted in FIG. 5, the primary imaging device 201 may use the color imager 202 to capture color 2D images of the patient's face from one or more different positions. For example, the color imager may capture images of the left side of the face from a left position, images of the right side of the face from a right side position, and of the front of the face from a front position. In some embodiments, the front, left, and right images may be captured by moving the camera into each different position and then capturing the image from that position.

In some embodiments, the color imager 202 may be sensitive to infrared light and may capture color images of the patient's face simultaneously with or synchronized with the capture of 3D image data of the patient's face by the imaging devices 206a, 206b, and 206c. The color imager 202, may capture 2D image data simultaneously or synchronized with the capture of the 3D image data. In some embodiments, the color imager 202, may capture 2D image data within 60 ms, less than 30 ms, or less than 10 ms of the capture of the 3D image data. The color data in the 2D image data of the patient's face may include IR data, such as the structured light from a light projector, as discussed herein. In some embodiments, the field of view of the color imager 202 may be calibrated with the imaging devices 206a, 206b, and 206c such that the filed of view of the color imager is in a known relationship with the imaging devices 206a, 206b, 206c. Capturing data simultaneously allows the 2D color image data generated by the color imager 202 to be combined with the 3D image data of the imaging devices 206a, 206b, and 206c without computationally intensive registration methods that attempt to align the 2D image data with the 3D image data. Instead, because the data is captured in close time proximity and movement of the imaging device is very small, the 2D image data and the 3D image data can be combined, such as based on the calibration of the image sensors.

In some embodiments, the camera may be stationary, such as coupled to or incorporated into the control unit 130, such as in a on or in a bezel of the monitor 132. The patient may move their head to look at the camera for a front image, look to the right to capture a left side image, and look to the left to capture a right side image of the patient's head and face. In some embodiments, image capture may be activated by receiving an input from the user, such as through a bottom on the extraoral imaging device 200. In some embodiments, a video or image stream form the image sensor may be generated and a facial detection algorithm may analyze the video or image stream and automatically capture the photos when the facial detention algorithm detects that the face and or head in a desired position. In some embodiments the user interface 134 may provide feedback to the patient on how to move their head and face to achieve the proper position. In some embodiments, audio feedback may be provided to guide the user into the proper position.

The color images may be captured using a 2D imaging device, such as a color CMOS sensor optically coupled to a lens such that the color imager has a focal length and field of view to capture the full face from a distance of between 50 cm and 100 cm, preferably as close as 75 cm.

The control unit 130 may determine that the desired photos have been captured and the user interface may update to indicate that 2D photo capture of the patient's face is complete and indicate that the process should proceed to the next step, such as capturing the 3D images of the face, capturing extraoral images of the patients arches, 3D scanning the patient's teeth with an intraoral scanner, or another process, as disclosed herein.

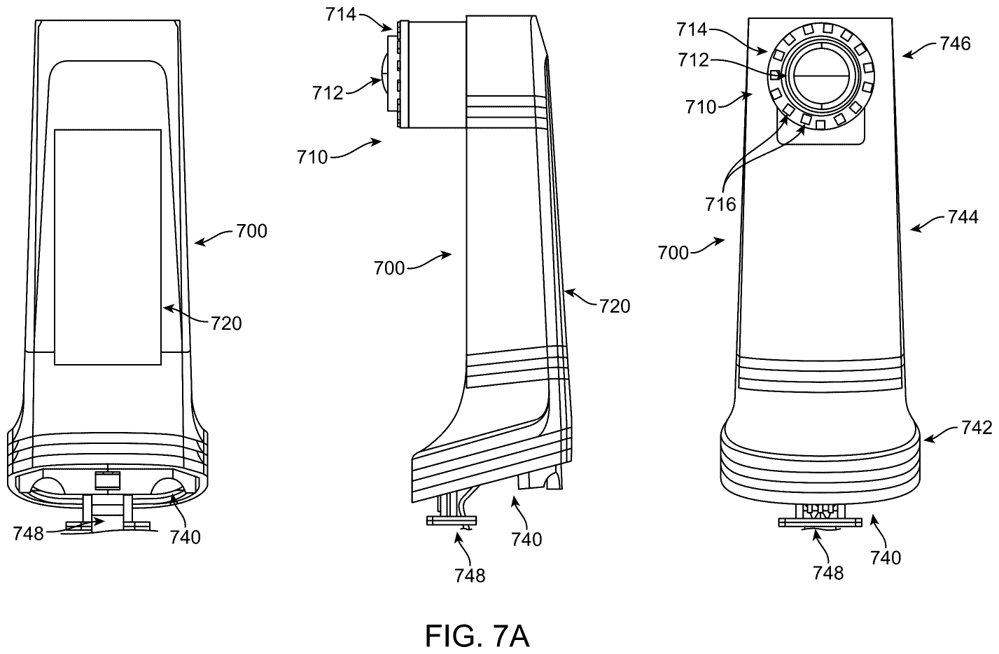

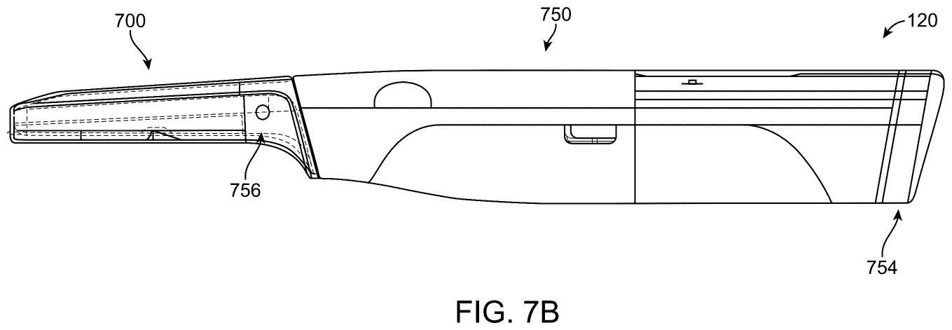

The control unit 130 may include a data store, such as data store 1022 of FIG. 7. The data store may include patient data, such as a patient data file that includes information for identifying or associating the data file with a particular patient, such as a name and birthdate, and for treating the patient. The treatment data may include a 3D intraoral scan data, such as that gathered by an intraoral scanner, a 3D face scan, 2D image data, extraoral 3D data, extraoral 2D data and other data described herein. At block 310, the control unit 130 may receive the 2D image data of the patient's face and then process the 2D image data, such as by formatting the 2D image data to conform with an expected format of 2D image data for the patient data file, in an automated matter, such as automatically after receiving the data. In some embodiments, the control unit 130 may process the data without additional input from an operator. In some embodiments, at block 310 the data is stored or otherwise associated with the patient data file in the datastore.

The light sources 208 may be configured to illuminate the face of the patient with at least twice the ambient illumination at a distance of at least 100 cm during capture of the 2D photos. For example, at least 1000 lux. In some embodiments, the light sources may illuminate the face with at least 1400 lux or at least 3000 lux. In some embodiments, the primary imaging unit 201 of the extraoral imaging device 200 may measure or the ambient light on the patient's face and adjust the light source to output at least twice the ambient light lux.

At block 320 the control unit 130 may activate the extraoral imaging device 200 for capturing 3D image data of the patient's face. The control unit 130 may display a prompt to the user to guide the user in capturing the 3D image data of the patient's face. For example, the display may include a user interface to receive input from the user indicating that the user is ready to capture the 3D image data of the patient's face.

As depicted in FIG. 5, the primary imaging device 201 may use the color imager IR imagers 206 to capture 3D data of the patient's face from one or more different positions. For example, the IR imagers 206 may capture 3D data of the left side of the face from a left position, 3D data of the right side of the face from a right side position, and of the front of the face from a front position. In some embodiments, the front, left, and right 3D data may be captured by moving the camera into each different position and then capturing the 3D data from that position.

In some embodiments, the camera may be stationary, such as coupled to or incorporated into the control unit 130, such as in a on or in a bezel of the monitor 132. The patient may move their head to look at the camera for a front 3D data, look to the right to capture a left side 3D data, and look to the left to capture a right side 3D data of the patient's head and face. In some embodiments, 3D data capture may be activated by receiving an input from the user, such as through a bottom on the extraoral imaging device 200. In some embodiments, a video or image stream form an image sensor, such as the color image sensor 202, may be generated and a facial detection algorithm may analyze the video or image stream and automatically capture the 3D data using the IR sensors when the facial detention algorithm detects that the face and or head in a desired position. In some embodiments the user interface 134 may provide feedback to the patient on how to move their head and face to achieve the proper position. In some embodiments, audio feedback may be provided to guide the user into the position.

The 3D data may be captured using a 3D imaging device, as described herein. For example, when capturing the 3D data, two cameras may work together to generate point clouds for the patient's face and head. The cameras may use structured light, time of flight, or other 3D imaging techniques to generate point clouds that represent the 3D location of the surfaces of the patient's face and head. The primary imaging unit 201 of the extraoral imaging device 200 may include multiple imaging devices, which may include both a light projector and light sensors, such as a CMOS imaging sensor, to capture the 3D data. The imaging devices 206a, 206b, and 206c, may capture 3D image data simultaneously, such as within less than 60 ms, less than 30 ms, or less than 10 ms. Capturing data simultaneously allows the point clouds generated from each of the cameras to be combined without computationally intensive registration methods that attempt to align two point clouds or other 3D data, such as a 3D model, together into one model or set of 3D data. Instead, because the data is captured in close time proximity and movement of the imaging device is very small, the 3D data from each simultaneously captured image may simply be combined based on the know geometric arrangement of the imaging devices 206a, 206b, and 206c.

In some embodiments, a stereoscopic pair of imagers such as stereoscopic pairs 210a, 210b may be used to generate point clouds. A stereoscopic pair of imagers may include a pair imaging devices in a known spatial relationship that simultaneously image an object from different angles. For example, stereoscopic pair 210a may include IR imager 206a and 206b while stereoscopic pair may include IR imagers 206b and 206c. In some embodiments, IR imagers 206a and 206c may comprise a third stereoscopic pair. The images from a stereoscopic pair may be used to generate a point cloud representing the 3D location of the surfaces of the objects in the field of view of each stereoscopic pair. Stereoscopic imaging may use photogrammetry or other 3D imaging processes to generate the point clouds.

In some embodiments, capturing extraoral 3D image data of the patient's face including one or more stereoscopic imaging pairs with the extraoral imaging device includes capturing the least two stereoscopic imaging pairs of a view of the patient's face within 30 ms of each other. In some embodiments, at least two stereoscopic imaging pairs of a view of the patient's face are captured simultaneously. Point clouds of 3D surface data of the patient's face from each stereoscopic imaging pair may be combined into a single point cloud. Because the point clouds were captured within close time proximity of each other, they may be combined without registration.

In some embodiments, point clouds generated form each of the view may be combined, such as though registration to generate a 3D model of the patient's face, including the left, front, and right sides.

The IR imager may include an IR light sensitive image sensor optically coupled to a lens such that the IR imager has a focal length and field of view to capture the full face from a distance of between 50 cm and 100 cm, preferably as close as 75 cm.

The IR imagers 206 may be arranged in a straight line or may be staggered such that each a line that passes though the centers of IR imager 206a and 206b interests with a line that passes through the centers of IR imager 206b and 206c. The distance between IR imager pairs may be the same, such as shown in FIG. 2A, or they may be different. For example, the distance between imager 206a and 206b may be different that the distance between imagers 206b and 206c.

In some embodiments, the actions at block 310 and block 320 may take place at the same time, their actions may be interleaved. For example, a right face 2D color image may be captured at the same time as right face 3D data, then center face 2D color image may be captured at the same time as center face 3D data, and then left face 2D color image may be captured at the same time as left face 3D data. In some embodiments, a right face 2D color image may be captured, then right face 3D data, then a center face 2D color image may be captured, then center face 3D data, then left face a 2D color image may be captured, and then left face 3D data.

The control unit 103 and the user interface may provide prompts for each of the captures and feedback to guide the data capture.

At block 320, the control unit 130 may receive the 3D image data of the patient's face and then process the 3D image data, such as by formatting the 3D image data to conform with an expected format of 3D image data for the patient data file, in an automated matter, such as automatically after receiving the data. In some embodiments, the control unit 130 may process the data without additional input from an operator. In some embodiments, at block 320 the data is stored or otherwise associated with the patient data file in the datastore.

At block 330 the control unit 130 may activate the extraoral imaging device 200 for capturing 2D image data of the patient's intraoral cavity. The control unit 130 may display a prompt to the user to guide the user in capturing the 2D image data of the patient's intraoral cavity. For example, the display may include a user interface to receive input from the user indicating that the user is ready to capture the 2D image data of the patient's intraoral cavity.

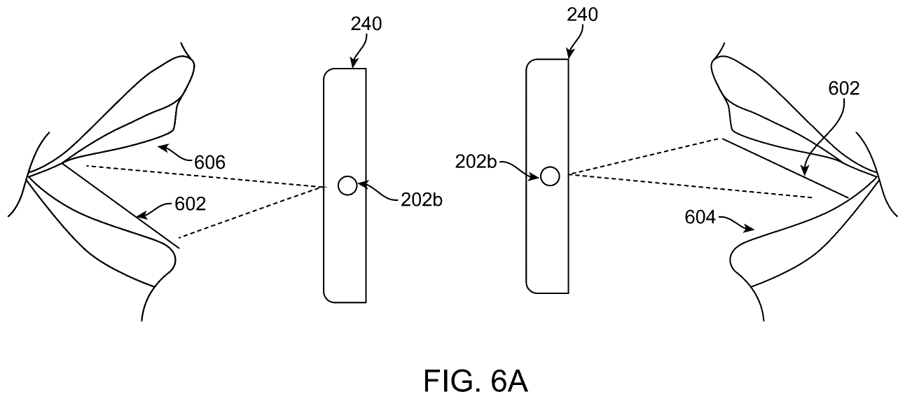

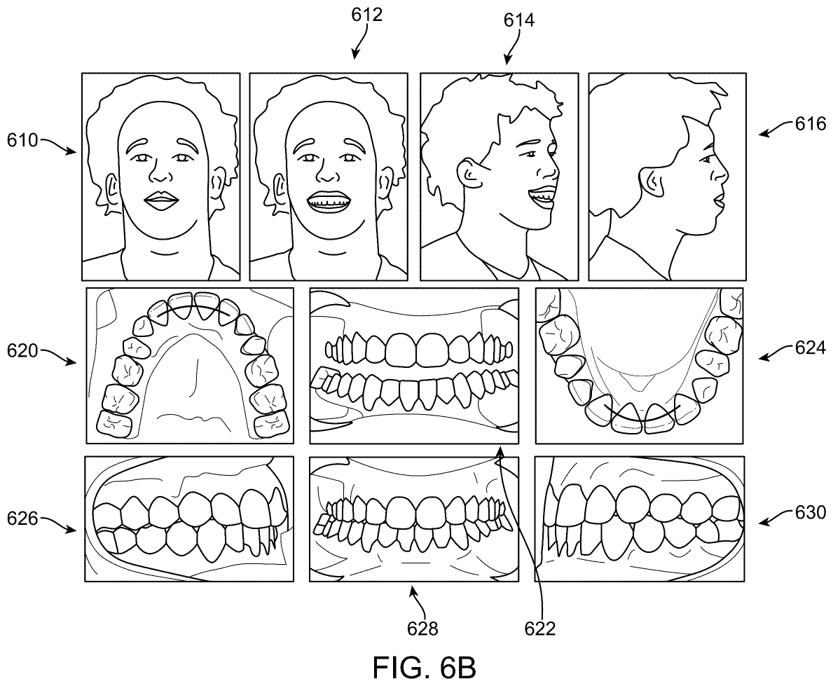

FIG. 6 depicts the use of the secondary imaging device 240 to capture extraoral images of the patient's dentition using the color imager 202b. In some embodiments, the primary imaging device 201 may be used to capture the extraoral images of the patient's dentition, for example, using the imager 202a. In some embodiments, the imager 202a may have a lens with multiple focal lengths, a first focal length to image the face and a second focal length to image the intraoral cavity.

The intraoral images of the patient's detention, such as the arches of the patient may be taking from an occlusal perspective. The occlusal perspective may be facilitated by the use of a mirror, such as mirror 602. To capture images of the upper arch, the mirror 602 may be placed within the oral cavity at an angle with the reflective surface pointed towards the upper arch 606, as depicted on the left side of FIG. 6. The secondary imaging unit 240 and its imager 202b may be pointed at the reflective surface to image the upper arch 606 of the patient from an occlusal perspective.

To capture images of the lower arch, the mirror 602 may be placed within the oral cavity at an angle with the reflective surface pointed towards the lower arch 604, as depicted on the right side of FIG. 6. The secondary imaging unit 240 and its imager 202b may be pointed at the reflective surface to image the upper arch 606 of the patient from an occlusal perspective.

In some embodiments, image capture of the patient's arch may be activated by receiving an input from the user, such as through a bottom on the extraoral imaging device 200. In some embodiments, a video or image stream form an image sensor, such as the color image sensor 202b, may be generated and a dental arch detection algorithm may analyze the video or image stream and automatically capture the 2D data using the image sensor 202b when the dental arch detection algorithm detects that the upper or lower arch a desired position and within the field of view. In some embodiments, the user interface 134 may provide feedback to the patient on how to move or position their mouth to achieve the proper position. In some embodiments, audio feedback may be provided to guide the user into the proper position.

At block 330, the control unit 130 may receive the 2D image data of the patient's intraoral cavity and then process the 2D image data, such as by formatting the 2D image data to conform with an expected format of 2D image data for the patient data file, in an automated matter, such as automatically after receiving the data. In some embodiments, the control unit 130 may process the data without additional input from an operator. In some embodiments, at block 330 the data is stored or otherwise associated with the patient data file in the datastore.

At block 340 the control unit 130 may activate the intraoral imaging device 200 for capturing 3D image data of the patient's intraoral cavity, including the dentition. The control unit 130 may display a prompt to the user to guide the user in capturing the 3D image data of the patient's intraoral cavity. For example, the display may include a user interface to receive input from the user indicating that the user is ready to capture the 3D image data of the patient's intraoral cavity.

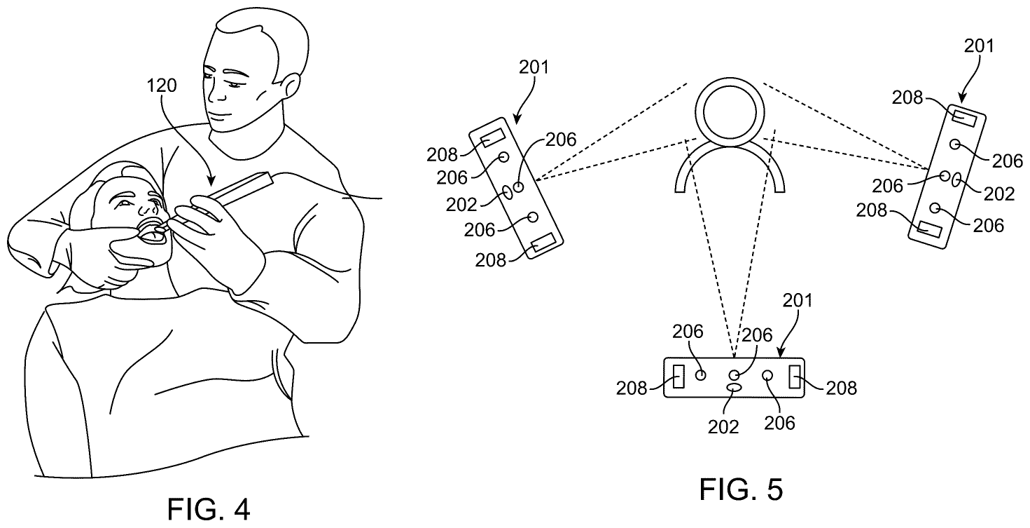

As depicted in FIG. 4, a dental professional may use the intraoral imaging device to scan the dentition of the patient, including the teeth and gingiva to generate the 3D model of the patient's dentition, as described herein.

At block 340, the control unit 130 may receive the 3D image data of the patient's intraoral cavity and then process the 3D image data, such as by formatting the 3D image data to conform with an expected format of 3D image data for the patient data file, in an automated matter, such as automatically after receiving the data. In some embodiments, the control unit 130 may process the data without additional input from an operator. In some embodiments, at block 330 the data is stored or otherwise associated with the patient data file in the datastore.

At block 350 the control unit 130 may prepare the 2D and 3D image data. The actions described herein at block 350 may also take place at one or more of blocks 310, 320, 330, and 340. For example, the 2D images of the patients face and intra oral cavity may be cropped to a predetermined image size, such as a predetermined image height and width in pixels. In some embodiments, the color image data from the color 2D images of the patient's face may be combined with the 3D date of the patient's face to generate a color 3D model of the patient's face. In some embodiments, the 3D model of the dentition may be combined with the color 3D model of the patient's face to generate an accurate 3D model and depiction of the patient's face and detention. The dynamic occlusion of the patient's teeth may be modeled based on the combined model. Dynamic occlusion is the interaction of the lower jaw as it moves relative to the upper jaw while the respective teeth of the jaws are in contact.

At block 360 the prepared data may be sent to a treatment planning system for generating orthodontic or prosthodontic treatment plans.

At block 370 the prepared data may be used to generate post treatment images of the patient. For example, after sending the prepared data to the treatment planning system, a treatment plan with a 3D model of the teeth in a final orthodontic position and/or with prosthodontics, such as a veneers, crowns, or bridges may received. The updated teeth model may be used with the 3D face model as an interactive mirror. For example, live video from the 2D imaging device 202 or live 3D data from the 3D imaging device 206 may be received by the control unity 130. The 3D model of the patient's face may be generated based on the position of the patient's face in the live video or 3D data. The 3D model of the patient's teeth from the treatment plan may be placed into the 3D model of the patient's face and displayed on the screen. In this way, the monitor 132 acts like a mirror, but instead of reflecting the patient's face, it shows the patient's face with their predicted post-treatment detention.

FIG. 6B depicts some of the views that a dental professional may capture with a 2D imaging device, such as a color camera. The views may include extraoral full-face views. The full-face views may include a frontal image of the patient's face with a natural closed mouth, capturing the relationship between the mouth, lips, and face. Such as depicted in image 610. The full-face views may also include a frontal image of the patient's face with a natural smile, capturing the relationship between the lips, teeth, and face. Such as depicted in image 612. These views may be useful for aesthetic assessments and to understand the overall facial symmetry.

The views may include profile face views. Profile face views may include images of the patient's face, which may be taken from both the left and right, to generate left and right profile images. Such as the left profile view depicted in 616. The profile views help in assessing the jawline, the relationship of the jaws to each other, and the overall facial profile. The profile views may include images of the patient with their natural smile and/or a closed mouth.

The views may include partial profile face views. Partial profile face views may include images of the patient's face, which may be taken from partially in front of the face from both the left and right, to generate left and right partial profile images. Such as the ¾ profile view depicted in 614. The partial profile views help in assessing the jawline, the relationship of the jaws to each other, and the overall facial profile. The profile views may include images of the patient with their natural smile and/or a closed mouth.

The views may include profile face views. Profile face views may include images of the patient's face, which may be taken from both the left and right, to generate left and right profile images. Such as the left profile view depicted in 616. The profile views help in assessing the jawline, the relationship of the jaws to each other, and the overall facial profile. The profile views may include images of the patient with their natural smile and/or a closed mouth.

The views may also include images of the dentition. For example, the views may include an anterior view, a right buccal view, a left buccal view, a maxillary occlusal view, and a mandibular occlusal view. The anterior view may include a frontal image showing the upper and lower front teeth with the upper and lower aches in occlusion, such as depicted in image 628 or non-occlusion, such as open. For example, as depicted in image 622. These views may aid in assessing alignment, symmetry, and the condition of the incisors and canines and other teeth.

The right buccal view may include an image of the right side of the mouth, showing the teeth in occlusion from the canine to the molars. In some embodiments, the image may include the right incisors to the right molars and may include the teeth in occlusion or not in occlusion. Image 626 depicts an example of a right buccal view with may aid in assessing the alignment, bite relationship, and the condition of the posterior teeth on the right side.

The left buccal view may include an image of the left side of the mouth, showing the teeth in occlusion from the canine to the molars. In some embodiments, the image may include the left incisors to the left molars and may include the teeth in occlusion or not in occlusion. Image 630 depicts an example of a left buccal view with may aid in assessing the alignment, bite relationship, and the condition of the posterior teeth on the left side.

The maxillary occlusal view may include an image looking up at the upper teeth of the maxillary arch from below. Imaging the maxillary occlusal view may be aided by a mirror, as discussed herein or by the patient tilting their head back to facilitate capturing the occlusal view of the upper arch. The maxillary occlusal view may be used to assess the arrangement, alignment, and condition of the upper teeth.

The mandibular occlusal view may include an image looking down at the lower teeth of the maxillary arch from above. Imaging the mandibular occlusal view may be aided by a mirror, as discussed herein or by the patient tilting their head down or forward to facilitate capturing the occlusal view of the lower arch. The mandibular occlusal view may be used to assess the arrangement, alignment, and condition of the lower teeth.

FIGS. 7A and 7B depict an imaging sleeve 700 for use with an intraoral scanner 120. The imaging sleeve 700 may include a cavity 740 in which a distal end 756 of the intraoral scanner 120 may be inserted.

The imaging sleeve 700 is configured to fit over the distal end of the intraoral scanner 120. The imaging sleeve 700 includes the electronic, mechanical, and optical systems for capturing 2D images when attached to the intraoral scanner 120.

The sleeve 700 includes a body that forms an outer shell around a cavity 740 in which a distal end 756 of the intraoral scanner 120 may be inserted. The body may be formed from various materials such as metal, plastic, rubber, or composite materials.

The outer surface of the body may be smooth or have surface roughness features to enhance grip, friction, or aesthetic appeal. The inner surface of the body, that forms the outer surface of the cavity and which may come into contact with the distal end of the intraoral scanner may be smooth or textured. The shape of the inner surface of the body may match the shape of the distal end of the intraoral scanner. In in some emblements, the inner surface of the sleeve may be shaped to have an offset from the shape of the distal end of the intraoral scanner to provide a clearance between the sleeve and the distal end of the probe. The clearance may be between 0.5 mm and 2 mm.

In some embodiments, the proximal end of the sleeve, which, as depicted in FIGS. 7A and 7B, include the opening to the cavity may include a planned or other meeting portion shaped to secure, and in some embodiments seal the sleeve against the intraoral scanner body.

In some embodiments, the proximal end of the sleeve may be shaped to couple or otherwise mechanically engage with the body of the intraoral scanner. For example, the proximal end of the sleeve may include a seal, such as o-ring, or gasket to aid in fluid containment or dust protection. In some embodiments, the proximal end of the sleeve may incorporate a coupling to mechanically coupled or engage with the body intraoral scanner. Mechanical coupling may include an o-ring, slots, clip, or other releasable coupling.

During use, the sleeve is aligned with the distal end of the intraoral scanner and slid over it. If the sleeve has a fastening mechanism, it is engaged once the sleeve is in the correct position. If the sleeve includes any flanges or coupling mechanisms, they may be used to secure the sleeve in place on the distal end of the intraoral scanner, to aid in preventing decoupling during operation.

The sleeve may also include an electrical and or electromechanical coupling 748. The coupling 748 may aid in mechanically securing the sleeve to the intraoral scanner as discussed herein. The coupling 748 may also provide electrical connection in communication between the components of the imaging sleeve and the intraoral scanner.

The imaging sleeve 700 may extend from a proximal end 742 to a distal end 746. In imaging system 710 may extend from the distal end 746 of the imaging sleeve 700. The imaging system 710 may include components for the focusing and capturing of light. The imaging system 710 may include one or more lenses 712 including an objective lens that receives light into the imaging system 710 and projects it towards the imaging sensor. The one or more lenses may also include focusing lenses or other optical elements that focus the light from the objective lens onto the imaging sensor. In some embodiments the objective lens and the focusing hands may be the same. The image sensor converts light received by the imaging system 710 into electrical signals. The imaging sensor may be made up of millions of tiny photo sites, called pixels, that capture photon and convert them to electrical signals. CCD (Charge-Coupled Device) and CMOS (Complementary Metal-Oxide-Semiconductor) and other types of imaging sensors may be used in the imaging system 710.

The imaging system 710 may also include the shutter such as a mechanical shutter or an electronic shutter. A mechanical shutter is a movable physical light barrier that opens and closes to control the exposure time of light on the imaging sensor. An electronic shutter controls exposure time by electronically turning the sensor on and off or by electronically starting and stopping the capture of photons on the photo sites.

The imaging system 710 may also include an illumination system 714. The illumination system 714 may include one or more light sources such as LEDs 716 that may provide additional light for illuminating the patient. Although the illumination system 714 is depicted as a rain of LEDs 716. In some embodiments, the illumination system may include a single LED, or two, three, or four LEDs or other light sources arranged around the objective lens 712. In some embodiments illumination system 714 may include one or more light sources on the body of the of the sleeve 700.

In some embodiments, the illumination system 714 may include polarizing filters the polarized light leaving the light sources. In some embodiments, the imaging system 710 may include one or more polarizing filters that polarize the light received by the objective lens. Such a polarizing filter may be placed in front of or behind the objective lens.

In some embodiments, the sleeve 700 may include one or more processors for controlling the imaging system 710. The processors may control aspects of the focusing of light on the image sensor, the shutter or the image sensor, the illumination system 714, and other aspects of the imaging system 710. In some embodiments, the control of the imaging system 710 may be separate from the control of the intraoral scanner. In some embodiments, the processors of the intraoral scanner may work in cooperation with one or more components of the imaging system to capture images. In some embodiments the processors of the intraoral scanner may more work with processors of sleeve 700 to control the imaging system 710.

During image capture, the intraoral scanner 120 with the sleeve 700 attached thereto may be held in a vertical orientation with the distal end of the sleeve 700 the above the proximal end of the intraoral scanners handle with the lens oriented to face towards the patient.

In some embodiments, the sleeve 700 may include a communication system for communicating directly with the control unit 130, such as a wired or wireless communication system. In some embodiments, the communication system of the sleeve 700 may communicate through the intraoral scanner 120 to the control system 130.

A prototype sleeve 700 with an imaging system 710 and accompanying processing communication systems was developed and tested. The prototype sleeve 700 used an Edmund 4 mm, f/8 UCi series fixed focal length lens with an On Semiconductor 13MP AR1335 sensor and control circuitry electrically coupled through 4 MIPI lanes to a local processor that then communicated to a host PC. The prototype sleeve included a ring of white LEDs surrounding the objective lens. The prototype sleeve was mounted on and iTero intraoral imaging scanning probe. Testing showed that the prototype was able to resolve features less than 200 μm in size at a distance that provided a field of view but included the entire mouth opening of an adult patient.

In some embodiments, the sleeve 700 may include one or more heating elements 720. The heating element may be a resistive heating element that may be turned on momentarily in order to heat a mirror that may be used during the image collection process for example mirror 602 depicted in FIG. 6A. The heating element may be turned on prior to capturing images using the mirror. After the heating element is turned on the mirror may be placed against the heating element to be warmed. Warming the mirror may aid in reducing or preventing fogging up the mirror due to the humidity of the oral cavity. The heating element may extend in a longitudinal direction, such as in a proximal-distal direction with a length greater than its width. The heating element may be rectangular or another shape. The heating element may be located on an opposite side of the sleeve from the imaging system 710. The heating element may be located on a side of the sleeve facing away from a patient when images of the patient are captured by the imaging system 710. The heating element may include a convection system incorporated therein that blows hot air over or through the heating element. A mirror or other object may be placed in the heated air stream to be heated.

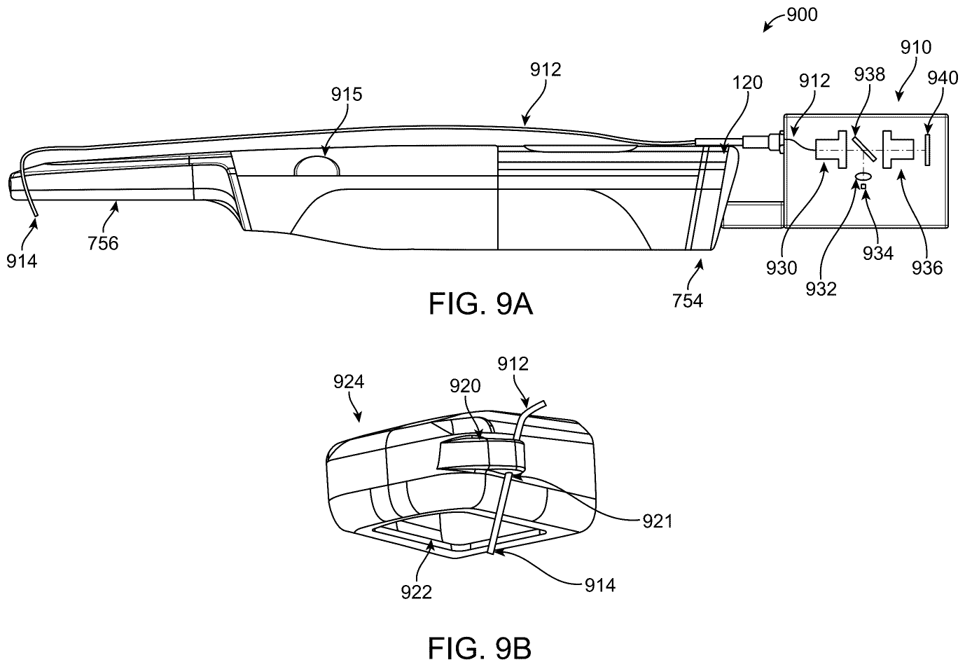

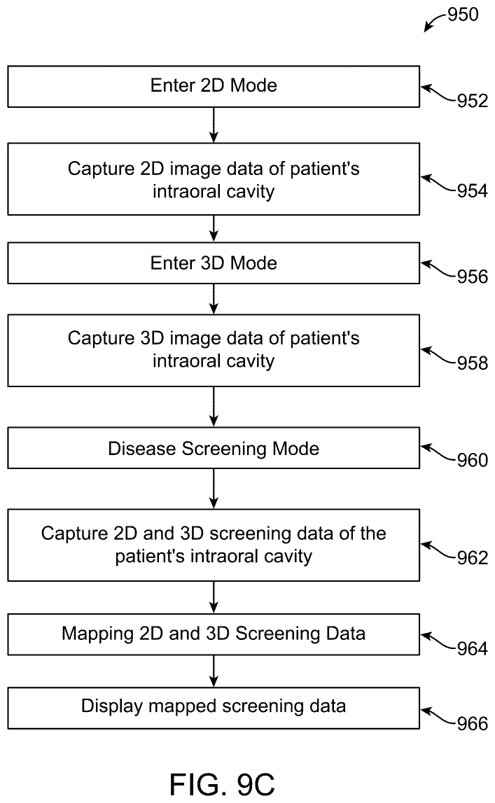

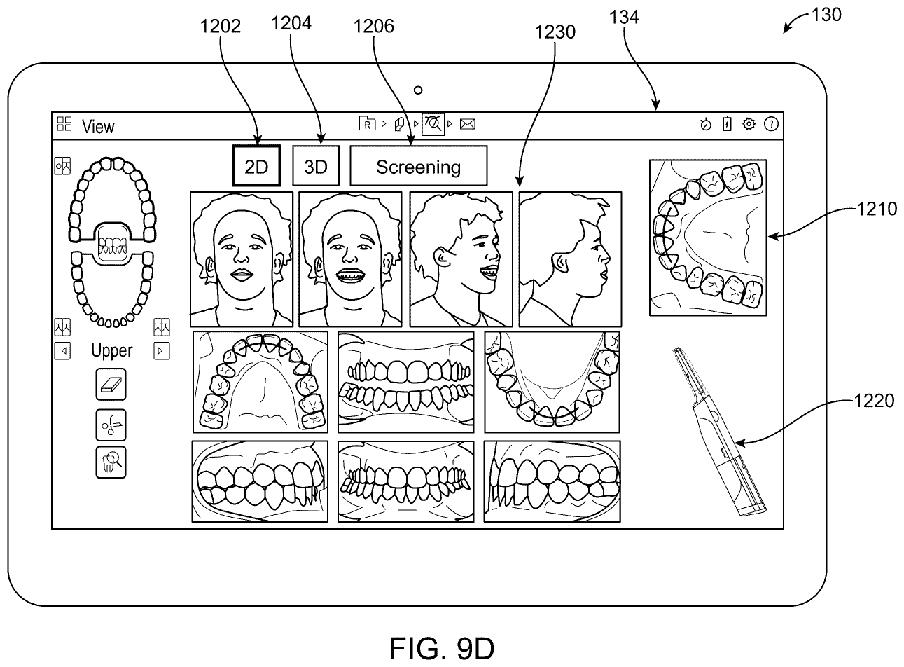

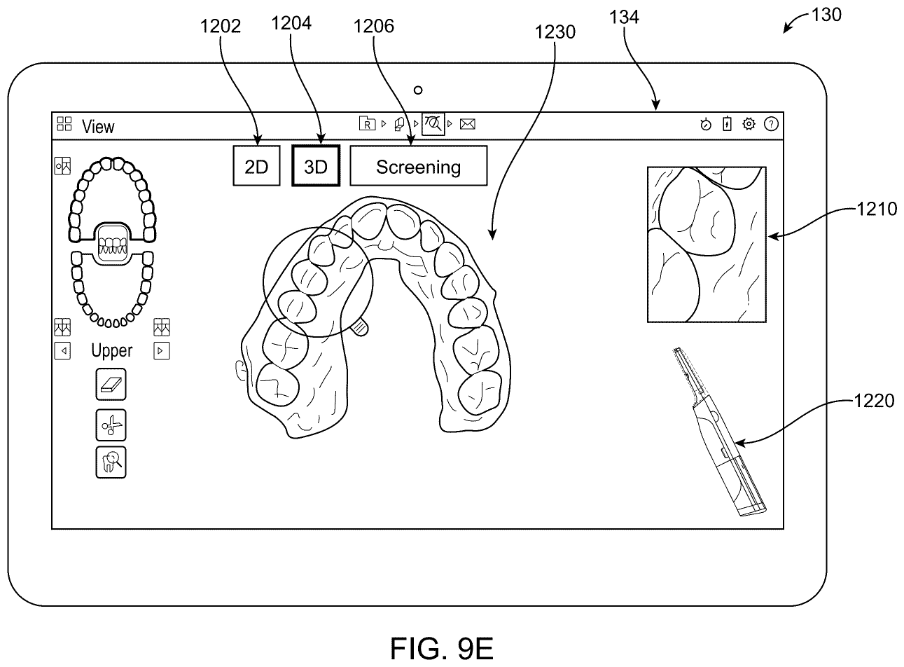

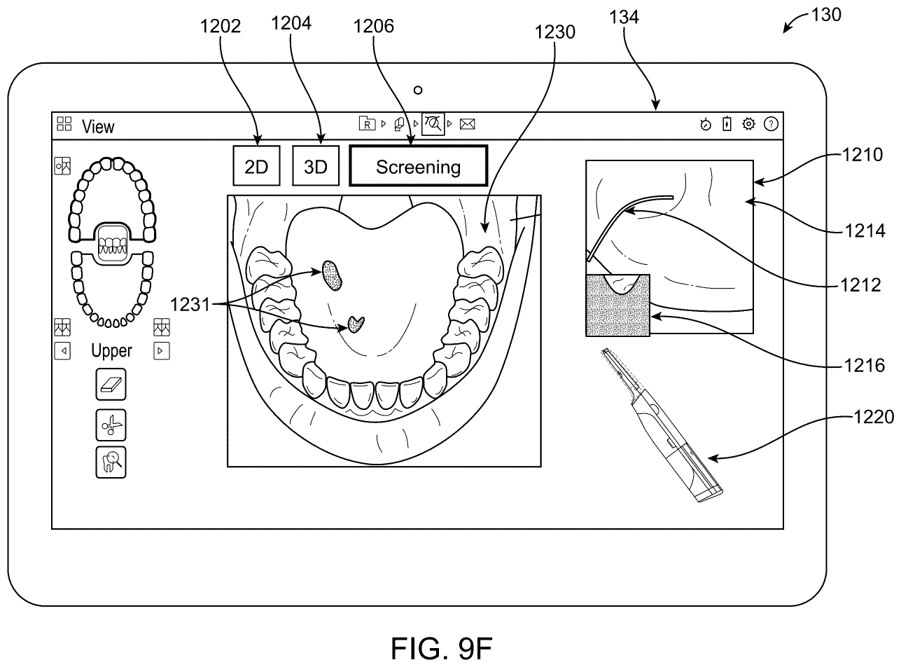

In some embodiments, the sleeve 700 may include a screen which may act as a viewfinder for the imaging system 710, displaying the current field of view of the imaging system and providing an interface for selecting an imaging mode, such as the 2D imaging mode, or other controls, such as exposure, pose, ect, such as though the interface by using the interface or aspects thereof of the interface shown and described with respect to FIG. 9D or elsewhere herein. In some embodiments, the screen may be located in place of the heating element 720.

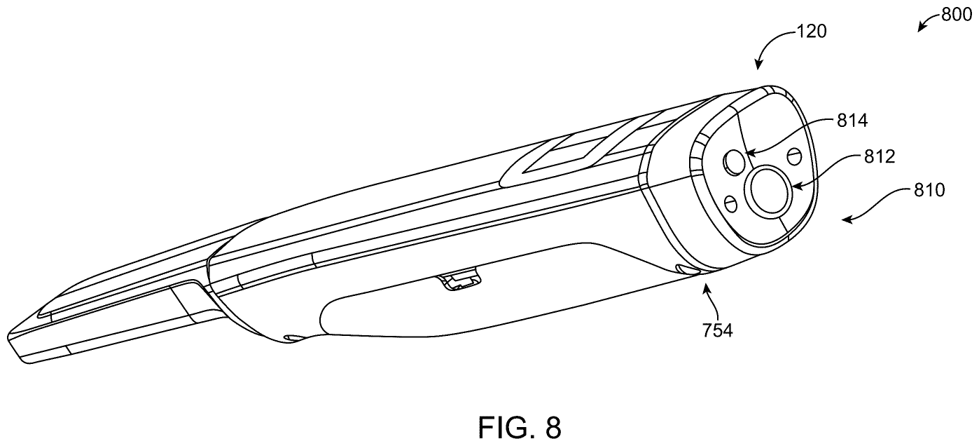

In some embodiments, an imaging system may be incorporated into a proximal and 754 of the intraoral scanner 120. For example, FIG. 8, depicts an imaging system 810 incorporated into the proximal end of the intraoral scanner 120 of the intraoral scanning system 800. The imaging system 810 is configured to fit within the proximal end of the intraoral scanner 120. The imaging system 810 includes the electronic, mechanical, and optical systems for capturing 2D images. In some embodiments, the imaging system 810 may be releasably mechanically and electronically coupled to the proximal end of the intraoral scanner 120. In some embodiments the imaging system may be incorporated into the proximal and of the intraoral scanning system.

In some embodiments, the distal end of the imaging system 810 may be shaped to couple or otherwise mechanically engage with the body of the intraoral scanner. For example, the distal end of the imaging system 810 may include a seal, such as o-ring, or gasket to aid in fluid containment or dust protection. In some embodiments, the distal end of the imaging system may incorporate a coupling to mechanically coupled or engage with the body intraoral scanner. Mechanical coupling may include an o-ring, slots, clip, or other releasable coupling.

During use, the imaging system 810 is aligned with the proximal end of the intraoral scanner and coupled to it. If the imaging system 810 has a fastening mechanism, it is engaged once the imaging system 810 is in the correct position. If the imaging system 810 includes any flanges or coupling mechanisms, they may be used to secure the sleeve in place on the distal end of the intraoral scanner, to aid in preventing decoupling during operation.

The imaging system 810 may also include an electrical and or electromechanical coupling. The coupling may aid in mechanically securing the imaging system 810 to the intraoral scanner as discussed herein. The coupling may also provide electrical connection in communication between the components of the imaging sleeve and the intraoral scanner.

The imaging system 810 may include components for the focusing and capturing of light. The imaging system 810 may include one or more lenses 812 including an objective lens that receives light into the imaging system 810 and projects it towards the imaging sensor. The one or more lenses may also include focusing lenses or other optical elements that focus the light from the objective lens onto the imaging sensor. In some embodiments the objective lens and the focusing hands may be the same. The image sensor converts light received by the imaging system 810 into electrical signals. The imaging sensor may be made up of millions of tiny photo sites, called pixels, that capture photon and convert them to electrical signals. CCD (Charge-Coupled Device) and CMOS (Complementary Metal-Oxide-Semiconductor) and other types of imaging sensors may be used in the imaging system 810.

The imaging system 810 may also include the shutter such as a mechanical shutter or an electronic shutter, as described herein.

The imaging system 810 may also include an illumination system 148. The illumination system 814 may include one or more light sources such as LEDs that may provide additional light for illuminating the patient. The illumination system may include a single LED, or two, three, or four LEDs or other light sources arranged around the objective lens 812. In some embodiments, illumination system 814 may include one or more light sources on the body of the of the imaging system 810 and/or the intraoral scanner body.

In some embodiments, the illumination system 814 may include polarizing filters the polarized light leaving the light sources. In some embodiments, the imaging system 810 may include one or more polarizing filters that polarize the light received by the objective lens. In some embodiments, the illumination system may be a direct illumination system wherein light is emitted directly towards the patient. In some embodiments, the illumination system may be an indirect illumination system wherein the light source emits light which is bounced off another surface before illuminating the patient. The surface may be incorporated into the sleeve or illumination system 814, the sleeve, or located elsewhere.

In some embodiments, the imaging system 810 may include one or more processors for controlling the imaging system 810. The processors may control aspects of the focusing of light on the image sensor, the shutter or the image sensor, the illumination system 814, and other aspects of the imaging system 810. In some embodiments, the control of the imaging system 810 may be separate from the control of the intraoral scanner. In some embodiments, the processors of the intraoral scanner may work in cooperation with one or more components of the imaging system to capture images. In some embodiments the processors of the intraoral scanner may more work with processors of the imaging system 810.

During image capture, the intraoral scanner 120 with the imaging system 810 at the proximal end may be held in a horizontal orientation with the proximal end of the intraoral scanner 120 having the imaging system may tend thereon oriented to face towards the patient.

In some embodiments, the imaging system 810 may include a communication system for communicating directly with the control unit 130, such as a wired or wireless communication system. In some embodiments, the communication system of the imaging system 810 may communicate through the intraoral scanner 120 to the control system 130.

FIGS. 9A and 9B depicts aspects of a system 900 for detecting oral cancer. Squamous cell carcinoma of most prevalent type of oral cancer. Most oral cancers are preceded by clinically evident oral potentially malignant disorders (OPMDs) and microscopically evident altered epithelial changes known as oral epithelial dysplasia (OED). The current gold standard to diagnose OED is biopsy and histopathology of suspicious oral lesions that are identified by conventional visual oral examination. However, it can be difficult even for experienced clinicians to choose when and where to biopsy. Many general practitioners lack expertise to distinguish OPMDs from clinically similar benign lesions. This creates challenges to the clinicians in determining which oral lesions are at highest risk to contain OED and require biopsy.

The system 900 aids in the detection and location of oral cancers in patients and identifying appropriate biopsy locations. The system 900 includes a modular intraoral scanner, such as intraoral scanner 120, a detection system 910 configured to be removably coupled to the proximal end 754 of the intraoral canner 120, and a fiber optic guide 920, which may be a part of the distal end 756 of the intraoral scanner 120 or a sleeve 924 that may be received on the distal end 756 of the intraoral scanner 120.

The system 900 locates the oral cancer using a combination of fluorescence of the oral cancer with the detection system 910 and location using the 3D imaging of the intraoral scanner. Fluorescence is the emission of light by a material when they are exposed to specific wavelengths of light, such as light in the ultraviolet (UV) to blue light spectrum. This phenomenon occurs because certain molecules, known as fluorophores, absorb the excitation light form a light source that emits light in the UV to blue light spectrum and then emits light at a longer wavelength.

In the context of oral cancers, fluorescence may be useful because a stain may be applied to the patient's tissue prior to screening or during the screening process before imaging with the screening system. The stain may be configured to target malignant and pre-malignant tissues while not staining healthy tissue or staining healthy tissue to a lesser extent than target malignant and pre-malignant tissues or other diseased.

Fluorescence Imaging is a non-invasive technique used to detect oral cancers and pre-cancerous lesions by taking advantage of the differences in fluorescence between healthy and abnormal tissues. Namely, that healthy tissue may appear as normal due to last of stain while diseased tissues, such as malignant or pre-malignant tissue may fluoresce when exposed to excitation illumination.

The detection system 910 includes a light source 932, which may emit light in the blue or violet light. The light source may be a blue LED. The system 910 is configured to illuminate portions of the oral cavity with the light from the light source. The oral tissue tissues absorb this light, and fluorophores within the tissues emit light at different wavelengths and intensities based on many factors, such as the presence or absence of oral cancers. The emitted fluorescence light may be received by the system and captured by a sensor 940, creating an image that highlights areas of altered fluorescence.

The light source 932 emits excitation light through a condenser lens 932. The light exits the lens 932 and is reflected off of a dichroic filter 938, through the lens 930, which may be a first objective lens associate with the fiber optic, into the fiber optic 912. The excitation light travels through the fiber optic 912, exiting at a distal end 914 and illuminating a patient's oral tissue. Light form the fluorescence of the oral tissue enters the distal end 914 of the fiber optic 912, travels down the fiber optic, though the lens 930, through the dichroic filter 938, the lens 936, which may be a second objective lens associated with the image sensor, and is then focused onto the image sensor 940, which captures an image of the light.

While the system 910 is capturing images of the oral tissue fluorescence or lack thereof, the 3D scanner of the intraoral scanner 120 is capturing 3D image data of the oral cavity. The distal end 914 of the fiber optic is visible within the field of view of the 3D scanner. The 3D scanner generates 3D scan data that includes both the tissue of the oral cavity and the distal end of the fiber optic while the detection system 910 captures 2D fluorescence data. The 2D fluorescence data may then be mapped to the 3D data based on the location of the distal end of the fiber optic relative to the oral tissue.

In some embodiments, the 3D scanner of the intraoral scanner 120 may have many modes. For example, the 3D scanner may have a first 3D scanning mode where tissue that moves during the scanning process is ignored or removed from the 3D model build using 3D scan data captured during the first mode. Such a mode is useful with generating a 3D model of hard tissue, such as the teeth, and of soft tissue that does not move, such as the gingiva near the teeth. Ignoring tissue that moves, such as tissue of the tongue or cheeks, aids in building the 3D model of the teeth and gingiva.

However, oral cancers may be found on moveable tissue, such as the tissue of the tongue and checks. The 3D scanner may have a second mode where moving tissue is not ignored when building a 3D model or when determining the location of the fiber optic, and/or field of view of the fiberoptic movable or moving tissue. The 3D scanner images the moving tissue to determine the 3D shape of the tissue and the location of the optical fiber while the detection system 910 images the fluorescence of the tissue. The system 900 then maps the fluorescence data onto the 3D data. In some embodiments, the controller 130 maps the data.

The system 900 may include a sleeve 924 shown in FIG. 9B that slides over, engages with, and/or covers the distal end of the intraoral scanner. The sleeve 924 may include an aperture or clear window 922 through which light may pass into the intraoral scanner for use in 3D scanning. The sleeve 924 may also include a fiber optic guide 920. The fiber optic guide 920 may include an aperture 921 through which the fiber optic 912 may pass. The fiber optic guide 920 engages with the fiber optic 912 and aids in maintaining the position of the fiber optic 912 during use. The position of the fiber optic 912 may be adjusted by sliding the fiber optic within the aperture.

FIGS. 9C-9F depict aspects of a method 950 and associated controls, displays, and feedback related to carrying out method 950. The method may include entering a 2D image capture mode at block 952, capturing 2D image data of patient's intraoral cavity at block 954, entering a 3D imaging mode at block 956, capturing 3D image data of a patient's intraoral cavity at block 958, entering a screening mode at block 960, capturing 2D and 3D screening data of the patient's intraoral cavity at block 962, mapping the 2D and 3D screening data at block 964, and displaying the mapped screening data at block 966.

The method 950, processes that take place at each block of the method 950, and the data captured and generated by the method 950 and the processes may be used in or with method 300, and vice versa. For example, the processes at each block of method 950 may take place as part of method 300 or the processes at each block of method 300 may take place as part of method 950. In some embodiments, the 2D data, 2D data, screening data, and/or mapped data of method 950 may be used with method 300. For example, the method 950 my capture or generate data which is then used at block 350, block 360, and/or block 370 of method 300. The capture of 2D and 3D data discussed in block 310, 320, 330, and 340, of method 300 may be captured in a respective 2D or 3D mode and/or capture process as described in method 950.

At block 952 the method 950 may include entering a 2D image capture mode. FIG. 9D shows aspects of a controller 130 with a user interface 134 for use with method 950. To enter the 2D imaging mode, a user may select the 2D imaging mode via the user interface 134. For example, the user, such as a dental professional, may interact with a button or other feature 1202 displayed on the screen to enter the 2D imaging mode. In some embodiments, the system may receive an input that causes the system 130 to enter the 2D image capture mode. For example, the system may receive an input or instruction via a button or other feature 1202 displayed on the screen. In some embodiments, a physical button or switch may be used to enter the 2D image capture mode. The button may be a button 915 on the intraoral scanner or a button on the controller 130.

Upon entering the 2D imaging mode, the user interface on the display may update to a 2D imaging mode interface. The interface may include a display object, such as a view finder window 1210 that displays an image of the current field of view of the 2D imaging device. The window may update at regular intervals, such as multiple times per second or continuously to provide the current field of view.

The user interface 134 may also include a display object, such as an attachment operation window 1220. The attachment operation window may include images for how to connect a 2D imaging attachment such as the imaging sleeve 700 onto the intraoral scanner. The attachment operation window 1220 may include an animation that shows how to connect the 2D imaging attachment onto the intraoral scanner. The images or animation may include depicting how to initially align the 2D imaging attachment such as the imaging sleeve 700 with the distal end of the intraoral scanner, how to slide the sleeve over the distal end of the intraoral scanner, and how to check and/or determine that the sleeve is properly fit over the distal end of the intraoral scanner. The user interface may display or otherwise provide feedback to indicate that the 2D imaging attachment is physically and/or electronically coupled to the intraoral scanner. For example, the attachment operation window 1220 may highlight a depiction of the 2D imaging attachment in a first color when it is not properly attached and in a second color when properly attached. In some embodiments, the button or other feature 1202 may change colors to indicate entering into the 2D image capture mode, to indicate that the 2D imaging attachment is not properly coupled to the intraoral scanner, and to indicate that the system is ready to begin capturing 2D images.

In some embodiments, the system may enter the 2D image capture mode upon detection of the 2D imaging attachment is physically and/or electronically coupled to the intraoral scanner. In some embodiments, the system may leave the 2D image capture mode upon detection of the 2D imaging attachment is not physically and/or electronically coupled to the intraoral scanner or removed from the intraoral scanner.

When entering and/or in the 2D image capture mode, the user interface 134 may include a 2D image guidance and image display element or window 1230. In this area, the user interface may include generic images, which may include drawings, that show the various 2D views of the patient that are to be captured. The user may select an image to be captured or the system may receive an input for an image to be captured.

At block 954 the method 950 may include capturing 2D image data of patient's intraoral cavity. The 2D imaging device may then start capturing images and displaying them in the viewfinder window 1210. The system 130 may detect the contents of the images and determining when an acceptable image is captured and then automatically save and display the image. The image may be confirmed or rejected by the user. If confirmed, then the system may automatically move to the next image to be captured or may receive input from the user on which image is to be captured next. In some embodiments, the system automatically moves to the next image to be captured unless or until the system receives input to retake an image. When an acceptable image is captured, the system may display the image in place of the image guidance image that showed the view of the patient that was to be captured. This process may repeat until all the 2D images are captured.

After the 2D images are captured, the attachment operation window 1220 may include images for how to disconnect a 2D imaging attachment such as the imaging sleeve 700 from the intraoral scanner. The attachment operation window 1220 may include an animation that shows how to disconnect the 2D imaging attachment from the intraoral scanner. The images or animation may include depicting how to mechanically and/or electrically disengage the 2D imaging attachment such as the imaging sleeve 700 from the distal end of the intraoral scanner, such as how to disengage a coupling or latch holding the two together, how to slide the sleeve off of the distal end of the intraoral scanner, and how to check and/or determine that the sleeve is properly stored, such as a storage position or cradle 138 on the controller 130. The user interface may display or otherwise provide feedback to indicate that the 2D imaging attachment is physically and/or electronically decoupled from the intraoral scanner. For example, the attachment operation window 1220 may highlight a depiction of the 2D imaging attachment in a first color when it is attached and in a second color when detached. In some embodiments, the button or other feature 1202 may change colors to indicate leaving the 2D image capture mode, to indicate that the 2D imaging attachment is decoupled from the intraoral scanner, and to indicate that the system is ready to enter another mode. In some embodiments, the system may automatically leave the 2D imaging mode upon detachment of the 2D imaging attachment from the intraoral scanner or placement of the 2D imaging device in a cradle or otherwise stowed.

At block 956 the method 950 may include entering a 3D image capture mode. FIG. 9E shows aspects of a controller 130 with a user interface 134 for use with method 950. To enter the 3D imaging mode, a user may select the 3D imaging mode via the user interface 134. For example, the user, such as a dental professional, may interact with a button or other feature 1204 displayed on the screen to enter the 3D imaging mode. In some embodiments, the system may receive an input that causes the system 130 to enter the 3D image capture mode. For example, the system may receive an input or instruction via a button or other feature 1204 displayed on the screen. In some embodiments, a physical button or switch may be used to enter the 3D image capture mode. The button may be a button 915 on the intraoral scanner or a button on the controller 130.

Upon entering the 3D imaging mode, the user interface on the display may update to a 3D imaging mode interface. The interface may include a display object, such as a view finder window 1210 that displays an image of the current field of view of the 3D imaging device, such as a 3D scanner, including n intraoral scanner 120. The window may update at regular intervals, such as multiple times per second or continuously to provide the current field of view.

The user interface 134 may also include a display object, such as an attachment operation window 1220. The attachment operation window may include images for how to disconnect an attachment, such as a 2D imaging attachment or screening attachment that should be removed from the intraoral canner before 3D scanning begins. The attachment operation window 1220 may include an animation that shows how to disconnect the attachment from the intraoral scanner. The images or animation may include depicting how to mechanically and/or electrically disengage the attachment from the distal or proximal end of the intraoral scanner, such as how to disengage a coupling or latch holding the two together, how to slide the attachment off of the distal end or proximal end of the intraoral scanner, and how to check and/or determine that the attachment is properly stored, such as a storage position or cradle 138 on the controller 130.

The user interface may display or otherwise provide feedback to indicate that the 2D imaging attachment is physically and/or electronically decoupled from the intraoral scanner. For example, the attachment operation window 1220 may highlight a depiction of the 2D imaging attachment in a first color when it is attached and in a second color when detached.

In some embodiments, the button or other feature 1202 may change colors to indicate entering into the 3D image capture mode, to indicate that no unneeded attachments are coupled to the intraoral scanner, and to indicate that the system is ready to begin capturing 3D image data.

In some embodiments, the system may enter the 3D image capture mode upon completion of the 2D image capture or completion of another capture mode. In some embodiments, the system may leave the 3D image capture mode upon detection of the 2D imaging attachment or another attachment is physically and/or electronically coupled to the intraoral scanner.

When entering and/or in the 3D image capture mode, the user interface 134 may include a 3D image guidance and image display element or window 1230. In this area, the user interface may include generic images, which may include drawings, that show which parts of the intraoral cavity of the patient that are to be captured, such as a generic dentition of the patient, including the upper and lower arches. The user may select one of the upper and lower arches to be captured or the system may receive an input for which portion of the intraoral cavity is to be captured.

In the 3D image capture mode, the system may be configured to ignore or remove captured 3D image data of moving tissue, such as the checks and tongue, when stitching the 3D image data into a 3D model.

At block 958 the method 950 may include capturing 3D image data of patient's intraoral cavity. The 3D imaging device, such as the intraoral scanner, may then start capturing images and displaying them in the viewfinder window 1210. The system 130 may display the 3D model as it is garnered from the captured 3D image data in the 3D image guidance and image display element or window 1230.

After the 3D image data is captured for a first arch, the user interface may automatically begin displaying guidance for the other arch or a user input may be received to display the guidance for the other arch. The guidance may include a starting location for the 3D scanning and a 3D scanning path along the arch or arches.

After 3D image data capture is complete, the system may automatically move to another mode or present a prompt to receive input for which mode to enter.

At block 960 the method 950 may include entering a screening mode. FIG. 9F shows aspects of a controller 130 with a user interface 134 for use with method 950. To enter the screening mode, a user may select the screening mode via the user interface 134. For example, the user, such as a dental professional, may interact with a button or other feature 1206 displayed on the screen to enter the screening mode. In some embodiments, the system may receive an input that causes the system 130 to enter the screening mode. For example, the system may receive an input or instruction via a button or other feature 1206 displayed on the screen. In some embodiments, a physical button or switch may be used to enter the screening mode. The button may be a button 915 on the intraoral scanner or a button on the controller 130.

Upon entering the screening mode, the user interface on the display may update to a screening mode interface. The interface may include a display object, such as a view finder window 1210 that displays an image of the current field of view 1214 of a 3D imaging device, such as a 3D scanner, which may include a 2D or 3D image of the tongue and the fiber optic 1212 and/or a current field of view 1216 of a 2D imaging device, such as the detection system 910 which may include the current fluorescence view from the fiber optic. The window may update at regular intervals, such as multiple times per second or continuously to provide the current field of view.

The user interface 134 may also include a display object, such as an attachment operation window 1220. The attachment operation window may include images for how to connect a screening attachment such as the detection system 910 onto the intraoral scanner. The attachment operation window 1220 may include an animation that shows how to connect the detection system 910 attachment onto the intraoral scanner. The images or animation may include depicting how to initially align the detection system 910 attachment such as the detection system 910 with the proximal end of the intraoral scanner, how to detection system 910 coupling into a receptacle on the distal end of the intraoral scanner, and how to check and/or determine that the detection system 910 is properly secured to the proximal end of the intraoral scanner. The user interface may display or otherwise provide feedback to indicate that the screening attachment is physically and/or electronically coupled to the intraoral scanner. For example, the attachment operation window 1220 may highlight a depiction of the screening attachment in a first color when it is not properly attached and in a second color when properly attached. In some embodiments, the button or other feature 1206 may change colors to indicate entering into the screening mode, to indicate that the screening attachment is not properly coupled to the intraoral scanner, and to indicate that the system is ready to begin screening, such as by simultaneously capturing 3D image data using the intraoral scanner and 2D image data, such as fluorescence data using the detection system 910.

In the screening mode 3D image data of moving tissue may not ignored when building a 3D model or when determining the location of the fiber optic, and/or field of view of the fiberoptic with respect to the movable or moving tissue. The 3D scanner images the moving tissue to determine the 3D shape of the tissue and the location of the optical fiber while the detection system 910 images the fluorescence of the tissue.

In some embodiments, the system may enter the screening mode upon detection of the screening attachment physically and/or electronically coupled to the intraoral scanner. In some embodiments, the system may leave the screening capture mode upon detection of the screening attachment not physically and/or electronically coupled to the intraoral scanner or removed from the intraoral scanner.

When entering and/or in the screening capture mode, the user interface 134 may include a screening guidance and image display element or window 1230. In this area, the user interface may include generic images, which may include drawings, that show a representation of the tissue to be captured, such as the tongue or checks depicted in FIG. 9F. The user may select a portion of the oral cavity from the images, such as the tongue to be screened or the system may receive an input for portion of the oral cavity to be screened.

At block 962 the method 950 may include capturing screening image data of patient's intraoral cavity. The 3D imaging device may and the screening device, such as the detection system 910 then start capturing image data, such as 3D image data and 2D image data and displaying one or both of them in the viewfinder window 1210.

At block 964 the system 900 then maps the fluorescence data onto the 3D data. In some embodiments, the controller 130 maps the data.

At block 966 the mapped data is displayed for example in image display element or window 1230. The displayed mapped data may include the fluorescence data mapped on a 3D model of the tissue, such as the tongue generated based on the 3D scan data. In some embodiments, areas of concern 1231, which may be locations where fluorescence or other data captured during the screening process indicates a disease or cancer may be located. In some embodiments, the mapped data may include the fluorescence data mapped only a 3D model of the tissue, such as a generic tongue or a simplified model of a tongue to aid in shown where cancerous or other diseased tissue may be located.

After screening is complete, the attachment operation window 1220 may include images for how to disconnect the screening attachment from the intraoral scanner. The attachment operation window 1220 may include an animation that shows how to disconnect the screening attachment from the intraoral scanner. The images or animation may include depicting how to mechanically and/or electrically disengage the screening attachment from the proximal end of the intraoral scanner, such as how to disengage a coupling or latch holding the two together, how to slide the attachment off of the proximal end of the intraoral scanner, and how to check and/or determine that the attachment is properly stored, such as a storage position or cradle 138 on the controller 130. The user interface may display or otherwise provide feedback to indicate that the screening attachment is physically and/or electronically decoupled from the intraoral scanner. For example, the attachment operation window 1220 may highlight a depiction of the screening attachment in a first color when it is attached and in a second color when detached. In some embodiments, the button or other feature 1202 may change colors to indicate leaving the screening mode, to indicate that the screening attachment is decoupled from the intraoral scanner, and to indicate that the system is ready to enter another mode. In some embodiments, the system may automatically leave the screening mode upon detachment of the screening attachment from the intraoral scanner or placement of the screening device in a cradle or otherwise stowed.

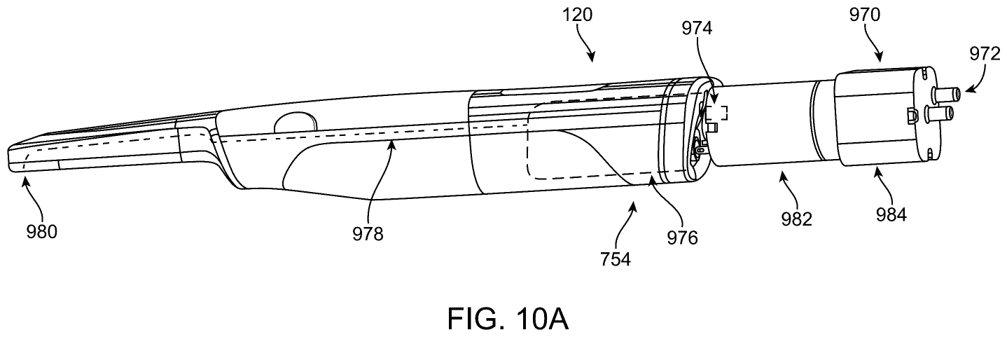

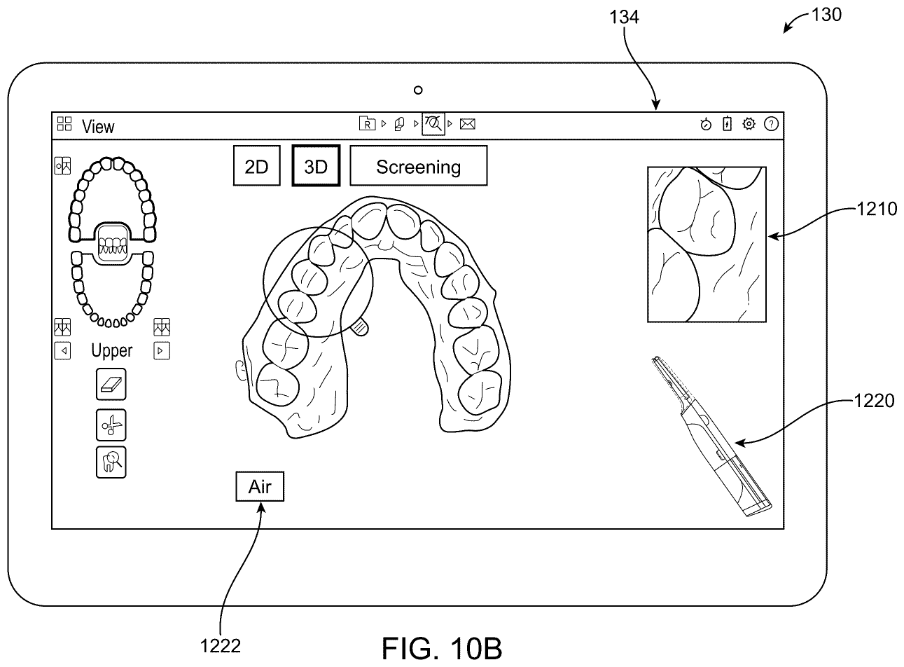

FIGS. 10A and 10B depict aspects of an air attachment and use thereof. In some embodiments, an air system may be incorporated into a proximal and 754 of the intraoral scanner 120. For example, FIG. 10A, depicts an air system 970 incorporated into the proximal end of the intraoral scanner 120. The imaging air system 970 is configured to fit within a cavity 976 of the proximal end of the intraoral scanner 120. The air system 970 includes the electronic and mechanical components for providing pressure air flow to an oral cavity via the intraoral cavity, such as an electric motor 982 and an air pump or fan 984. In some embodiments, the air system 970 may be releasably mechanically and electronically coupled to the proximal end of the intraoral scanner 120. In some embodiments the imaging system may be incorporated into the proximal and of the intraoral scanning system.

In some embodiments, the body of the air system may be shaped to couple or otherwise mechanically engage with the body of the intraoral scanner. For example, the distal end of the air system 970 may include a seal, such as o-ring, or gasket to aid mechanically and fluidically connecting the air system 970 to the intraoral scanner.

During use, the power may be supplied to the air system 970 by the intraoral scanner, such as from a battery or other electrical energy storage device within the intraoral scanner. The electrical energy drives an electric motor 982 attached to an air pump 984 which draws air from an inlet 972, increases the energy in the air, and expels the air out of an outlet 974. The energized air, which is at a higher pressure than the air at the inlet travels down a conduit or tube 978 to the distal end of the intraoral scanner and out a nozzle 980 located proximate the 3D imaging device at the distal end of the intraoral scanner. The air from the air system is expelled out the nozzle 980 and may be used to clear or dry one or more tissues of the intraoral cavity of a patient, such as during 2D or 3D image capture.

For example, FIG. 10B shows aspects of a controller 130 with a user interface 134 for use with the air system and the methods discussed herein. To enter an air supply mode, a user may select the air supply mode via the user interface 134. For example, the user, such as a dental professional, may interact with a button or other feature 1222 displayed on the screen to enter the air supply mode. In some embodiments, the system may receive an input that causes the system 130 to enter the air supply mode. For example, the system may receive an input or instruction via a button or other feature 1222 displayed on the screen. The button may be a button 915 on the intraoral scanner or a button on the controller 130.

Upon entering the air supply mode, the user interface on the display may update to an air supply interface, which may be an addition to one or more of the other interfaces disused herein.

The user interface 134 may include a display object, such as an attachment operation window 1220. The attachment operation window may include images for how to connect an air supply attachment such as the air system 970 onto the intraoral scanner. The attachment operation window 1220 may include an animation that shows how to connect the air system 970 attachment into the intraoral scanner. The images or animation may include depicting how to initially align the air system 970 attachment with the proximal end of the intraoral scanner, how to slide the air system 970 into the cavity in the proximal end of the intraoral scanner, and how to check and/or determine that the air system 970 is properly fit within the proximal end of the intraoral scanner. The user interface may display or otherwise provide feedback to indicate that the air system 970 attachment is physically and/or electronically coupled to the intraoral scanner. For example, the attachment operation window 1220 may highlight a depiction of the air system 970 attachment in a first color when it is not properly attached and in a second color when properly attached. In some embodiments, the button or other feature 1222 may change colors to indicate entering into the air system 970 mode, to indicate that the air system 970 attachment is not properly coupled to the intraoral scanner, and to indicate that the system is ready to begin supplying air. In some embodiments, after entering the air supply mode and while in the 3D scanning mode, the user may input a command and the system may receive the input to begin supplying air, for example though a button on the intraoral scanner or on the user interface, as described herein.

In some embodiments, the system may enter the air supply mode upon detection of the air supply attachment physically and/or electronically coupled to the intraoral scanner. In some embodiments, the system may leave the air supply mode upon detection of the air supply attachment not physically and/or electronically coupled to the intraoral scanner or removed from the intraoral scanner.

After the air supply is no longer needed, the attachment operation window 1220 may include images for how to disconnect the air supply attachment from the intraoral scanner. The attachment operation window 1220 may include an animation that shows how to disconnect the air supply attachment from the intraoral scanner. The images or animation may include depicting how to mechanically and/or electrically disengage the air supply attachment from the proximal end of the intraoral scanner, such as how to disengage a coupling or latch holding the two together, how to slide the air supply out of the proximal end of the intraoral scanner, and how to check and/or determine that the air system is properly stored, such as a storage position or cradle 138 on the controller 130. The user interface may display or otherwise provide feedback to indicate that the air supply attachment is physically and/or electronically decoupled from the intraoral scanner. For example, the attachment operation window 1220 may highlight a depiction of the air supply attachment in a first color when it is attached and in a second color when detached. In some embodiments, the button or other feature 1202 may change colors to indicate leaving the air supply mode, to indicate that the air supply attachment is decoupled from the intraoral scanner, and to indicate that the system is ready to enter another mode. In some embodiments, the system may automatically leave the air supply mode upon detachment of the air supply attachment from the intraoral scanner or placement of the air supply system in a cradle or otherwise stowed.

### Computing System

FIG. 7 is a block diagram of an example computing system 1010 capable of implementing one or more of the embodiments described and/or illustrated herein. For example, all or a portion of computing system 1010 may perform and/or be a means for performing, either alone or in combination with other elements, one or more of the steps described herein (such as one or more of the steps illustrated in 1-6). All or a portion of computing system 1010 may also perform and/or be a means for performing any other steps, methods, or processes described and/or illustrated herein.

Computing system 1010 broadly represents any single or multi-processor computing device or system capable of executing computer-readable instructions. Examples of computing system 1010 include, without limitation, workstations, laptops, client-side terminals, servers, distributed computing systems, handheld devices, or any other computing system or device. In its most basic configuration, computing system 1010 may include at least one processor 1014 and a system memory 1016.

Processor 1014 generally represents any type or form of physical processing unit (e.g., a hardware-implemented central processing unit) capable of processing data or interpreting and executing instructions. In certain embodiments, processor 1014 may receive instructions from a software application or module. These instructions may cause processor 1014 to perform the functions of one or more of the example embodiments described and/or illustrated herein.

System memory 1016 generally represents any type or form of volatile or non-volatile storage device or medium capable of storing data and/or other computer-readable instructions. Examples of system memory 1016 include, without limitation, Random Access Memory (RAM), Read Only Memory (ROM), flash memory, or any other suitable memory device. Although not required, in certain embodiments computing system 1010 may include both a volatile memory unit (such as, for example, system memory 1016) and a non-volatile storage device (such as, for example, primary storage device 1032, as described in detail below). In one example, software, such as instructions for execution by a processor for carrying out the methods of any of FIGS. 1-6, may be loaded into system memory 1016.

In some examples, system memory 1016 may store and/or load an operating system 1040 for execution by processor 1014. In one example, operating system 1040 may include and/or represent software that manages computer hardware and software resources and/or provides common services to computer programs and/or applications on computing system 1010. Examples of operating system 1040 include, without limitation, LINUX, JUNOS, MICROSOFT WINDOWS, WINDOWS MOBILE, MAC OS, APPLE'S IOS, UNIX, GOOGLE CHROME OS, GOOGLE'S ANDROID, SOLARIS, variations of one or more of the same, and/or any other suitable operating system.

In certain embodiments, example computing system 1010 may also include one or more components or elements in addition to processor 1014 and system memory 1016. For example, as illustrated in FIG. 7, computing system 1010 may include a memory controller 1018, an Input/Output (I/O) controller 1020, and a communication interface 1022, each of which may be interconnected via a communication infrastructure 1012. Communication infrastructure 1012 generally represents any type or form of infrastructure capable of facilitating communication between one or more components of a computing device. Examples of communication infrastructure 1012 include, without limitation, a communication bus (such as an Industry Standard Architecture (ISA), Peripheral Component Interconnect (PCI), PCI Express (PCIe), or similar bus) and a network.

Memory controller 1018 generally represents any type or form of device capable of handling memory or data or controlling communication between one or more components of computing system 1010. For example, in certain embodiments memory controller 1018 may control communication between processor 1014, system memory 1016, and I/O controller 1020 via communication infrastructure 1012.

I/O controller 1020 generally represents any type or form of module capable of coordinating and/or controlling the input and output functions of a computing device. For example, in certain embodiments I/O controller 1020 may control or facilitate transfer of data between one or more elements of computing system 1010, such as processor 1014, system memory 1016, communication interface 1022, display adapter 1026, input interface 1030, and storage interface 1034.

As illustrated in FIG. 7, computing system 1010 may also include at least one display device 1024 coupled to I/O controller 1020 via a display adapter 1026. Display device 1024 generally represents any type or form of device capable of visually displaying information forwarded by display adapter 1026. Similarly, display adapter 1026 generally represents any type or form of device configured to forward graphics, text, and other data from communication infrastructure 1012 (or from a frame buffer, as known in the art) for display on display device 1024.

As illustrated in FIG. 7, example computing system 1010 may also include at least one input device 1028 coupled to I/O controller 1020 via an input interface 1030. Input device 1028 generally represents any type or form of input device capable of providing input, either computer or human generated, to example computing system 1010. Examples of input device 1028 include, without limitation, a keyboard, a pointing device, a speech recognition device, variations or combinations of one or more of the same, and/or any other input device.

Additionally or alternatively, example computing system 1010 may include additional I/O devices. For example, example computing system 1010 may include I/O device 1036. In this example, I/O device 1036 may include and/or represent a user interface that facilitates human interaction with computing system 1010. Examples of I/O device 1036 include, without limitation, a computer mouse, a keyboard, a monitor, a printer, a modem, a camera, a scanner, a microphone, a touchscreen device, variations or combinations of one or more of the same, and/or any other I/O device.

Communication interface 1022 broadly represents any type or form of communication device or adapter capable of facilitating communication between example computing system 1010 and one or more additional devices. For example, in certain embodiments communication interface 1022 may facilitate communication between computing system 1010 and a private or public network including additional computing systems. Examples of communication interface 1022 include, without limitation, a wired network interface (such as a network interface card), a wireless network interface (such as a wireless network interface card), a modem, and any other suitable interface. In at least one embodiment, communication interface 1022 may provide a direct connection to a remote server via a direct link to a network, such as the Internet. Communication interface 1022 may also indirectly provide such a connection through, for example, a local area network (such as an Ethernet network), a personal area network, a telephone or cable network, a cellular telephone connection, a satellite data connection, or any other suitable connection.

In certain embodiments, communication interface 1022 may also represent a host adapter configured to facilitate communication between computing system 1010 and one or more additional network or storage devices via an external bus or communications channel. Examples of host adapters include, without limitation, Small Computer System Interface (SCSI) host adapters, Universal Serial Bus (USB) host adapters, Institute of Electrical and Electronics Engineers (IEEE) 1394 host adapters, Advanced Technology Attachment (ATA), Parallel ATA (PATA), Serial ATA (SATA), and External SATA (eSATA) host adapters, Fibre Channel interface adapters, Ethernet adapters, or the like. Communication interface 1022 may also allow computing system 1010 to engage in distributed or remote computing. For example, communication interface 1022 may receive instructions from a remote device or send instructions to a remote device for execution.

In some examples, system memory 1016 may store and/or load a network communication program 1038 for execution by processor 1014. In one example, network communication program 1038 may include and/or represent software that enables computing system 1010 to establish a network connection 1042 with another computing system (not illustrated in FIG. 7) and/or communicate with the other computing system by way of communication interface 1022. In this example, network communication program 1038 may direct the flow of outgoing traffic that is sent to the other computing system via network connection 1042. Additionally or alternatively, network communication program 1038 may direct the processing of incoming traffic that is received from the other computing system via network connection 1042 in connection with processor 1014.

Although not illustrated in this way in FIG. 7, network communication program 1038 may alternatively be stored and/or loaded in communication interface 1022. For example, network communication program 1038 may include and/or represent at least a portion of software and/or firmware that is executed by a processor and/or Application Specific Integrated Circuit (ASIC) incorporated in communication interface 1022.

As illustrated in FIG. 7, example computing system 1010 may also include a primary storage device 1032 and a backup storage device 1033 coupled to communication infrastructure 1012 via a storage interface 1034. Storage devices 1032 and 1033 generally represent any type or form of storage device or medium capable of storing data and/or other computer-readable instructions. For example, storage devices 1032 and 1033 may be a magnetic disk drive (e.g., a so-called hard drive), a solid state drive, a floppy disk drive, a magnetic tape drive, an optical disk drive, a flash drive, or the like. Storage interface 1034 generally represents any type or form of interface or device for transferring data between storage devices 1032 and 1033 and other components of computing system 1010. In one example, digital models of teeth and/or images of teeth may be stored and/or loaded in primary storage device 1032.

In certain embodiments, storage devices 1032 and 1033 may be configured to read from and/or write to a removable storage unit configured to store computer software, data, or other computer-readable information. Examples of suitable removable storage units include, without limitation, a floppy disk, a magnetic tape, an optical disk, a flash memory device, or the like. Storage devices 1032 and 1033 may also include other similar structures or devices for allowing computer software, data, or other computer-readable instructions to be loaded into computing system 1010. For example, storage devices 1032 and 1033 may be configured to read and write software, data, or other computer-readable information. Storage devices 1032 and 1033 may also be a part of computing system 1010 or may be a separate device accessed through other interface systems.

Many other devices or subsystems may be connected to computing system 1010. Conversely, all of the components and devices illustrated in FIG. 7 need not be present to practice the embodiments described and/or illustrated herein. The devices and subsystems referenced above may also be interconnected in different ways from that shown in FIG. 7. Computing system 1010 may also employ any number of software, firmware, and/or hardware configurations. For example, one or more of the example embodiments disclosed herein may be encoded as a computer program (also referred to as computer software, software applications, computer-readable instructions, or computer control logic) on a computer-readable medium. The term “computer-readable medium,” as used herein, generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The computer-readable medium containing the computer program may be loaded into computing system 1010. All or a portion of the computer program stored on the computer-readable medium may then be stored in system memory 1016 and/or various portions of storage devices 1032 and 1033. When executed by processor 1014, a computer program loaded into computing system 1010 may cause processor 1014 to perform and/or be a means for performing the functions of one or more of the example embodiments described and/or illustrated herein. Additionally or alternatively, one or more of the example embodiments described and/or illustrated herein may be implemented in firmware and/or hardware. For example, computing system 1010 may be configured as an Application Specific Integrated Circuit (ASIC) adapted to implement one or more of the example embodiments disclosed herein.

FIG. 8 is a block diagram of an example network architecture 1100 in which client systems 1110, 1120, and 1130 and servers 1140 and 1145 may be coupled to a network 1150. As detailed above, all or a portion of network architecture 1100 may perform and/or be a means for performing, either alone or in combination with other elements, one or more of the steps disclosed herein (such as one or more of the steps illustrated in FIGS. 1-6). All or a portion of network architecture 1100 may also be used to perform and/or be a means for performing other steps and features set forth in the instant disclosure.

Client systems 1110, 1120, and 1130 generally represent any type or form of computing device or system, such as example computing system 1010 in FIG. 7. Similarly, servers 1140 and 1145 generally represent computing devices or systems, such as application servers or database servers, configured to provide various database services and/or run certain software applications. Network 1150 generally represents any telecommunication or computer network including, for example, an intranet, a WAN, a LAN, a PAN, or the Internet.

As illustrated in FIG. 8, one or more storage devices 1160(1)-(N) may be directly attached to server 1140. Similarly, one or more storage devices 1170(1)-(N) may be directly attached to server 1145. Storage devices 1160(1)-(N) and storage devices 1170(1)-(N) generally represent any type or form of storage device or medium capable of storing data and/or other computer-readable instructions. In certain embodiments, storage devices 1160(1)-(N) and storage devices 1170(1)-(N) may represent Network-Attached Storage (NAS) devices configured to communicate with servers 1140 and 1145 using various protocols, such as Network File System (NFS), Server Message Block (SMB), or Common Internet File System (CIFS).

Servers 1140 and 1145 may also be connected to a Storage Area Network (SAN) fabric 1180. SAN fabric 1180 generally represents any type or form of computer network or architecture capable of facilitating communication between a plurality of storage devices. SAN fabric 1180 may facilitate communication between servers 1140 and 1145 and a plurality of storage devices 1190(1)-(N) and/or an intelligent storage array 1195. SAN fabric 1180 may also facilitate, via network 1150 and servers 1140 and 1145, communication between client systems 1110, 1120, and 1130 and storage devices 1190(1)-(N) and/or intelligent storage array 1195 in such a manner that devices 1190(1)-(N) and array 1195 appear as locally attached devices to client systems 1110, 1120, and 1130. As with storage devices 1160(1)-(N) and storage devices 1170(1)-(N), storage devices 1190(1)-(N) and intelligent storage array 1195 generally represent any type or form of storage device or medium capable of storing data and/or other computer-readable instructions.

In certain embodiments, and with reference to example computing system 1010 of FIG. 7, a communication interface, such as communication interface 1022 in FIG. 7, may be used to provide connectivity between each client system 1110, 1120, and 1130 and network 1150. Client systems 1110, 1120, and 1130 may be able to access information on server 1140 or 1145 using, for example, a web browser or other client software. Such software may allow client systems 1110, 1120, and 1130 to access data hosted by server 1140, server 1145, storage devices 1160(1)-(N), storage devices 1170(1)-(N), storage devices 1190(1)-(N), or intelligent storage array 1195. Although FIG. 8 depicts the use of a network (such as the Internet) for exchanging data, the embodiments described and/or illustrated herein are not limited to the Internet or any particular network-based environment.

In at least one embodiment, all or a portion of one or more of the example embodiments disclosed herein may be encoded as a computer program and loaded onto and executed by server 1140, server 1145, storage devices 1160(1)-(N), storage devices 1170(1)-(N), storage devices 1190(1)-(N), intelligent storage array 1195, or any combination thereof. All or a portion of one or more of the example embodiments disclosed herein may also be encoded as a computer program, stored in server 1140, run by server 1145, and distributed to client systems 1110, 1120, and 1130 over network 1150.

As detailed above, computing system 1010 and/or one or more components of network architecture 1100 may perform and/or be a means for performing, either alone or in combination with other elements, one or more steps of an example method for virtual care.

While the foregoing disclosure sets forth various embodiments using specific block diagrams, flowcharts, and examples, each block diagram component, flowchart step, operation, and/or component described and/or illustrated herein may be implemented, individually and/or collectively, using a wide range of hardware, software, or firmware (or any combination thereof) configurations. In addition, any disclosure of components contained within other components should be considered example in nature since many other architectures can be implemented to achieve the same functionality.

In some examples, all or a portion of the example systems disclosed herein may represent portions of a cloud-computing or network-based environment. Cloud-computing environments may provide various services and applications via the Internet. These cloud-based services (e.g., software as a service, platform as a service, infrastructure as a service, etc.) may be accessible through a web browser or other remote interface. Various functions described herein may be provided through a remote desktop environment or any other cloud-based computing environment.

In various embodiments, all or a portion of the example systems disclosed herein may facilitate multi-tenancy within a cloud-based computing environment. In other words, the software modules described herein may configure a computing system (e.g., a server) to facilitate multi-tenancy for one or more of the functions described herein. For example, one or more of the software modules described herein may program a server to enable two or more clients (e.g., customers) to share an application that is running on the server. A server programmed in this manner may share an application, operating system, processing system, and/or storage system among multiple customers (i.e., tenants). One or more of the modules described herein may also partition data and/or configuration information of a multi-tenant application for each customer such that one customer cannot access data and/or configuration information of another customer.

According to various embodiments, all or a portion of the example systems disclosed herein may be implemented within a virtual environment. For example, the modules and/or data described herein may reside and/or execute within a virtual machine. As used herein, the term “virtual machine” generally refers to any operating system environment that is abstracted from computing hardware by a virtual machine manager (e.g., a hypervisor). Additionally or alternatively, the modules and/or data described herein may reside and/or execute within a virtualization layer. As used herein, the term “virtualization layer” generally refers to any data layer and/or application layer that overlays and/or is abstracted from an operating system environment. A virtualization layer may be managed by a software virtualization solution (e.g., a file system filter) that presents the virtualization layer as though it were part of an underlying base operating system. For example, a software virtualization solution may redirect calls that are initially directed to locations within a base file system and/or registry to locations within a virtualization layer.

In some examples, all or a portion of the example systems disclosed herein may represent portions of a mobile computing environment. Mobile computing environments may be implemented by a wide range of mobile computing devices, including mobile phones, tablet computers, e-book readers, personal digital assistants, wearable computing devices (e.g., computing devices with a head-mounted display, smartwatches, etc.), and the like. In some examples, mobile computing environments may have one or more distinct features, including, for example, reliance on battery power, presenting only one foreground application at any given time, remote management features, touchscreen features, location and movement data (e.g., provided by Global Positioning Systems, gyroscopes, accelerometers, etc.), restricted platforms that restrict modifications to system-level configurations and/or that limit the ability of third-party software to inspect the behavior of other applications, controls to restrict the installation of applications (e.g., to only originate from approved application stores), etc. Various functions described herein may be provided for a mobile computing environment and/or may interact with a mobile computing environment.

In addition, all or a portion of the example systems disclosed herein may represent portions of, interact with, consume data produced by, and/or produce data consumed by one or more systems for information management. As used herein, the term “information management” may refer to the protection, organization, and/or storage of data. Examples of systems for information management may include, without limitation, storage systems, backup systems, archival systems, replication systems, high availability systems, data search systems, virtualization systems, and the like.

In some embodiments, all or a portion of the example systems disclosed herein may represent portions of, produce data protected by, and/or communicate with one or more systems for information security. As used herein, the term “information security” may refer to the control of access to protected data. Examples of systems for information security may include, without limitation, systems providing managed security services, data loss prevention systems, identity authentication systems, access control systems, encryption systems, policy compliance systems, intrusion detection and prevention systems, electronic discovery systems, and the like.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments, one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

FIG. 9 illustrates an exemplary tooth repositioning appliance 1100, such as an aligner that can be worn by a patient in order to achieve an incremental repositioning of individual teeth 1102 in the jaw. The appliance can include a shell (e.g., a continuous polymeric shell or a segmented shell) having teeth-receiving cavities that receive and resiliently reposition the teeth. An appliance or portion(s) thereof may be indirectly fabricated using a physical model of teeth. For example, an appliance (e.g., polymeric appliance) can be formed using a physical model of teeth and a sheet of suitable layers of polymeric material. The physical model (e.g., physical mold) of teeth can be formed through a variety of techniques, including 3D printing. The appliance can be formed by thermoforming the appliance over the physical model. In some embodiments, a physical appliance is directly fabricated, e.g., using additive manufacturing techniques, from a digital model of an appliance. In some embodiments, the physical appliance may be created through a variety of direct formation techniques, such as 3D printing. An appliance can fit over all teeth present in an upper or lower jaw, or less than all of the teeth. The appliance can be designed specifically to accommodate the teeth of the patient (e.g., the topography of the tooth-receiving cavities matches the topography of the patient's teeth), and may be fabricated based on positive or negative models of the patient's teeth generated by impression, scanning, and the like. Alternatively, the appliance can be a generic appliance configured to receive the teeth, but not necessarily shaped to match the topography of the patient's teeth. In some cases, only certain teeth received by an appliance will be repositioned by the appliance while other teeth can provide a base or anchor region for holding the appliance in place as it applies force against the tooth or teeth targeted for repositioning. In some cases, some or most, and even all, of the teeth will be repositioned at some point during treatment. Teeth that are moved can also serve as a base or anchor for holding the appliance as it is worn by the patient. In some embodiments, no wires or other means will be provided for holding an appliance in place over the teeth. In some cases, however, it may be desirable or necessary to provide individual attachments or other anchoring elements 1104 on teeth 1102 with corresponding receptacles or apertures 1106 in the appliance 1100 so that the appliance can apply a selected force on the tooth. Exemplary appliances, including those utilized in the Invisalign® System, are described in numerous patents and patent applications assigned to Align Technology, Inc. including, for example, in U.S. Pat. Nos. 6,450,807, and 5,975,893, as well as on the company's website, which is accessible on the World Wide Web (see, e.g., the URL “invisalign.com”). Examples of tooth-mounted attachments suitable for use with orthodontic appliances are also described in patents and patent applications assigned to Align Technology, Inc., including, for example, U.S. Pat. Nos. 6,309,215 and 6,830,450.

FIG. 10 illustrates a tooth repositioning system 1350 including a plurality of appliances 1253A, 1253B, 1253C. Any of the appliances described herein can be designed and/or provided as part of a set of a plurality of appliances used in a tooth repositioning system. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 1350 can include a first appliance 1253A corresponding to an initial tooth arrangement, one or more intermediate appliances 1253B corresponding to one or more intermediate arrangements, and a final appliance 1253C corresponding to a target arrangement. A target tooth arrangement can be a planned final tooth arrangement selected for the patient's teeth at the end of all planned orthodontic treatment. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

Optionally, in cases involving more complex movements or treatment plans, it may be beneficial to utilize auxiliary components (e.g., features, accessories, structures, devices, components, and the like) in conjunction with an orthodontic appliance. Examples of such accessories include but are not limited to elastics, wires, springs, bars, arch expanders, palatal expanders, twin blocks, occlusal blocks, bite ramps, mandibular advancement splints, bite plates, pontics, hooks, brackets, headgear tubes, springs, bumper tubes, palatal bars, frameworks, pin-and-tube apparatuses, buccal shields, buccinator bows, wire shields, lingual flanges and pads, lip pads or bumpers, protrusions, divots, and the like. In some embodiments, the appliances, systems and methods described herein include improved orthodontic appliances with integrally formed features that are shaped to couple to such auxiliary components, or that replace such auxiliary components.

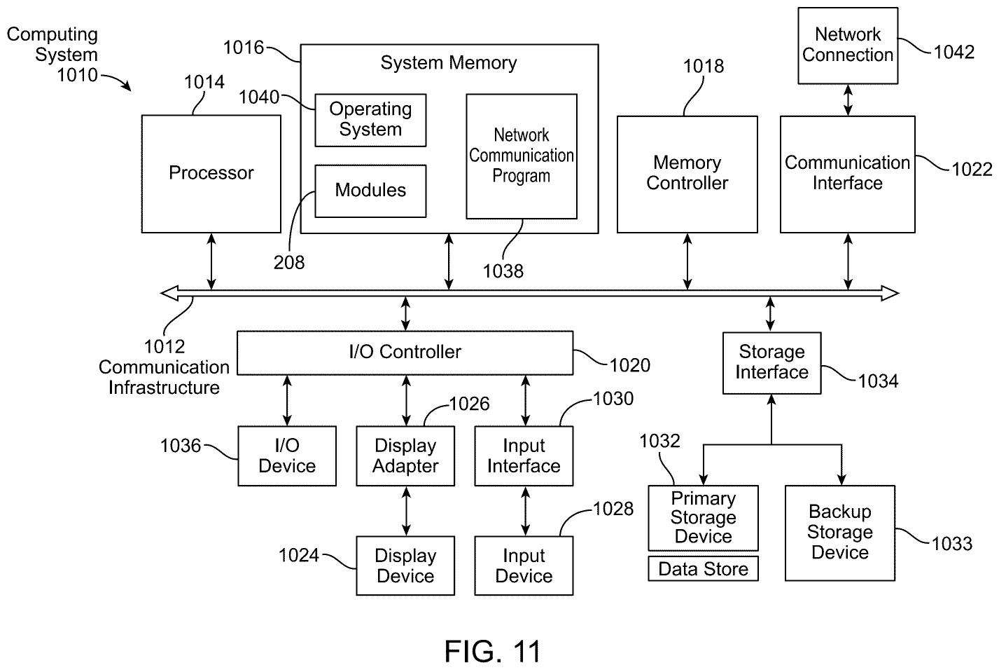

FIG. 11 illustrates a method 1300 of orthodontic treatment using a plurality of appliances, in accordance with many embodiments. The method 1300 can be practiced using any of the appliances or appliance sets described herein. In step 1310, a first orthodontic appliance is applied to a patient's teeth in order to reposition the teeth from a first tooth arrangement to a second tooth arrangement. In step 1320, a second orthodontic appliance is applied to the patient's teeth in order to reposition the teeth from the second tooth arrangement to a third tooth arrangement. The method 1300 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

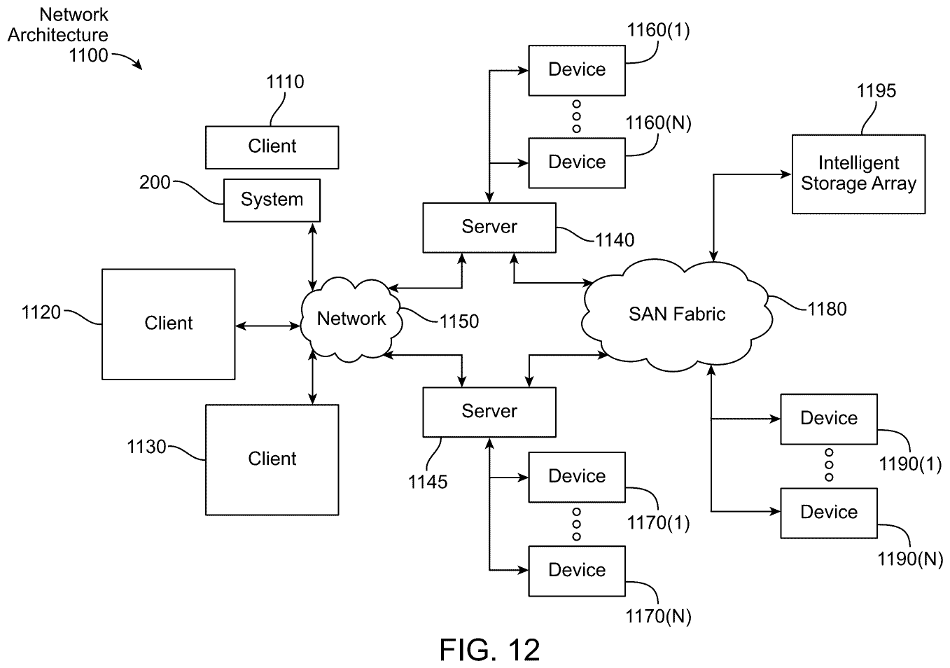

FIG. 12 illustrates a method 1400 for digitally planning an orthodontic treatment and/or design or fabrication of an appliance, in accordance with many embodiments. The method 1400 can be applied to any of the treatment procedures described herein and can be performed by any suitable data processing system. Any embodiment of the appliances described herein can be designed or fabricated using the method 1400.

In step 1410, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In step 1420, one or more treatment stages are generated based on the digital representation of the teeth. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In step 1430, at least one orthodontic appliance is fabricated based on the generated treatment stages. For example, a set of appliances can be fabricated to be sequentially worn by the patient to incrementally reposition the teeth from the initial arrangement to the target arrangement. Some of the appliances can be shaped to accommodate a tooth arrangement specified by one of the treatment stages. Alternatively or in combination, some of the appliances can be shaped to accommodate a tooth arrangement that is different from the target arrangement for the corresponding treatment stage. For example, as previously described herein, an appliance may have a geometry corresponding to an overcorrected tooth arrangement. Such an appliance may be used to ensure that a suitable amount of force is expressed on the teeth as they approach or attain their desired target positions for the treatment stage. As another example, an appliance can be designed in order to apply a specified force system on the teeth and may not have a geometry corresponding to any current or planned arrangement of the patient's teeth.

In some instances, staging of various arrangements or treatment stages may not be necessary for design and/or fabrication of an appliance. As illustrated by the dashed line in FIG. 12, design and/or fabrication of an orthodontic appliance, and perhaps a particular orthodontic treatment, may include use of a representation of the patient's teeth (e.g., receive a digital representation of the patient's teeth 1410), followed by design and/or fabrication of an orthodontic appliance based on a representation of the patient's teeth in the arrangement represented by the received representation.

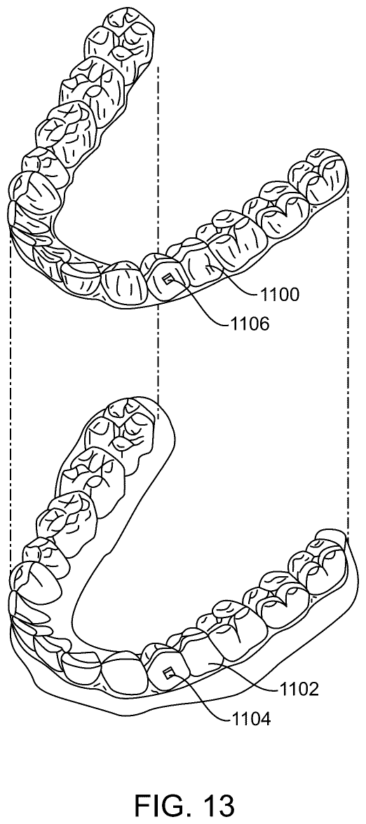

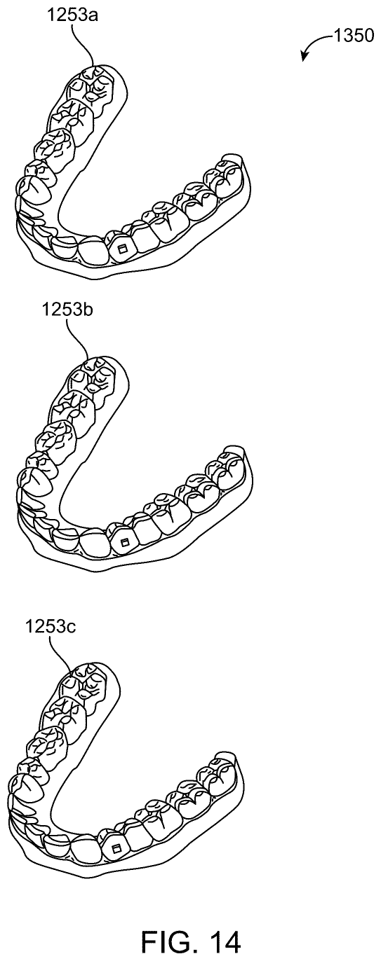

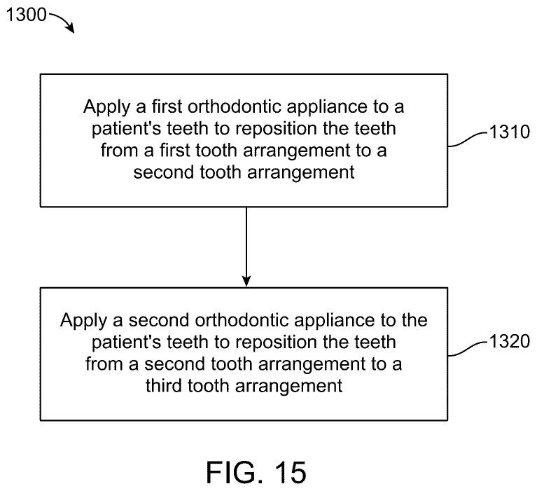

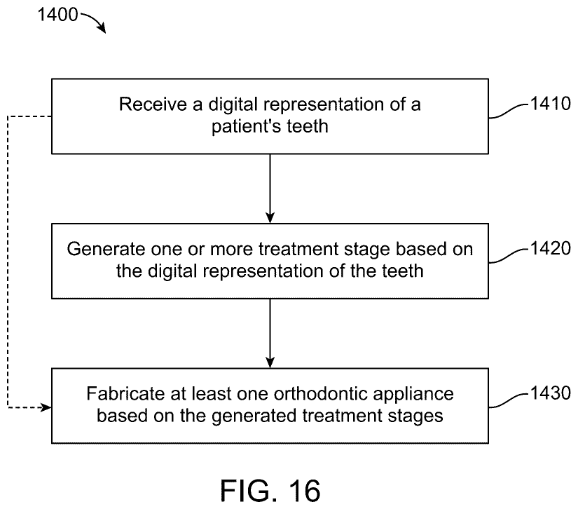

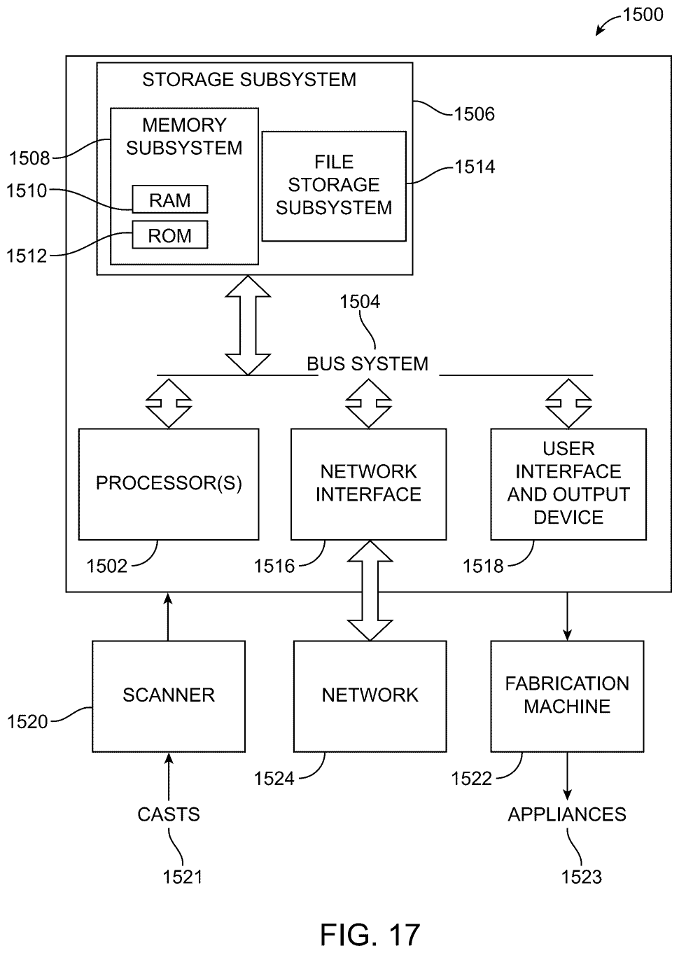

FIG. 13 is a simplified block diagram of a data processing system 1500 that may be used in executing methods and processes described herein and may incorporate aspects of the systems depicted in FIGS. 9 and 10 or may be part of the systems depicted in FIGS. 9 and 10. The data processing system 1500 typically includes at least one processor 1502 that communicates with one or more peripheral devices via bus subsystem 1504. These peripheral devices typically include a storage subsystem 1506 (memory subsystem 1508 and file storage subsystem 1514), a set of user interface input and output devices 1518, and an interface to outside networks 1516. This interface is shown schematically as “Network Interface” block 1516, and is coupled to corresponding interface devices in other data processing systems via communication network interface 1524. Data processing system 1500 can include, for example, one or more computers, such as a personal computer, workstation, mainframe, laptop, and the like.

The user interface input devices 1518 are not limited to any particular device, and can typically include, for example, a keyboard, pointing device, mouse, scanner, interactive displays, touchpad, joysticks, etc. Similarly, various user interface output devices can be employed in a system of the invention, and can include, for example, one or more of a printer, display (e.g., visual, non-visual) system/subsystem, controller, projection device, audio output, and the like.

Storage subsystem 1506 maintains the basic required programming, including computer readable media having instructions (e.g., operating instructions, etc.), and data constructs. The program modules discussed herein are typically stored in storage subsystem 1506. Storage subsystem 1506 typically includes memory subsystem 1508 and file storage subsystem 1514. Memory subsystem 1508 typically includes a number of memories (e.g., RAM 1510, ROM 1512, etc.) including computer readable memory for storage of fixed instructions, instructions and data during program execution, basic input/output system, etc. File storage subsystem 1514 provides persistent (non-volatile) storage for program and data files, and can include one or more removable or fixed drives or media, hard disk, floppy disk, CD-ROM, DVD, optical drives, and the like. One or more of the storage systems, drives, etc. may be located at a remote location, such coupled via a server on a network or via the internet/World Wide Web. In this context, the term “bus subsystem” is used generically so as to include any mechanism for letting the various components and subsystems communicate with each other as intended and can include a variety of suitable components/systems that would be known or recognized as suitable for use therein. It will be recognized that various components of the system can be, but need not necessarily be at the same physical location, but could be connected via various local-area or wide-area network media, transmission systems, etc.

Scanner 1520 includes any means for obtaining a digital representation (e.g., images, surface topography data, etc.) of a patient's teeth (e.g., by scanning physical models of the teeth such as casts 1521, by scanning impressions taken of the teeth, or by directly scanning the intraoral cavity), which can be obtained either from the patient or from treating professional, such as an orthodontist, and includes means of providing the digital representation to data processing system 1500 for further processing. Scanner 1520 may be located at a location remote with respect to other components of the system and can communicate image data and/or information to data processing system 1500, for example, via a network interface 1524. Fabrication system 1522 fabricates appliances 1523 based on a treatment plan, including data set information received from data processing system 1500. Fabrication machine 1522 can, for example, be located at a remote location and receive data set information from data processing system 1500 via network interface 1524.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters, such as numerals, refer to like elements. The present disclosure includes the following numbered clauses.

Clause 1. A dental scanning system, the system comprising: an intraoral scanner; an extraoral imaging device including a color imaging camera and a plurality of 3D imaging cameras configured to capture at least two stereoscopic image pairs; and non-transitory computer readable medium with instructions that, when executed by a processor, cause the system to carry out a method, the method including: capturing 2D image data of a patient's face with the extraoral imaging device; capturing extraoral 3D image data of the patient's face including one or more stereoscopic imaging pairs with the extraoral imaging device; capturing intraoral 3D image data of a dentition of the patient with the intraoral scanner; generating a 3D model of the patient's face and teeth using the 2D image data, the extraoral 3D image data, and the intraoral 3D image data.

Clause 2. The dental scanning system of clause 1, further comprising: a control unit including the processor and the non-transitory computer readable medium, and wherein the intraoral scanner and the extraoral imaging device are coupled to the control unit.

Clause 3. The dental scanning system of clause 2, further comprising: a portable cart, wherein the control unit, the extraoral imaging device, and the intraoral scanner are coupled to the portable cart.

Clause 4. The dental scanning system of clause 3, further comprising: a display coupled to the cart and the control unit.

Clause 5. The dental scanning system of clause 1, wherein capturing the capturing extraoral 3D image data of the patient's face including one or more stereoscopic imaging pairs with the extraoral imaging device includes capturing the least two stereoscopic imaging within 30 ms of each other.

Clause 6. The dental scanning system of clause 1, wherein capturing the capturing extraoral 3D image data of the patient's face including one or more stereoscopic imaging pairs with the extraoral imaging device includes capturing the least two stereoscopic imaging simultaneously.

Clause 7. The dental scanning system of clause 1, further comprising: generating a pair of point clouds from each stereoscopic imaging pair; and combining each respective pair of point clouds to generate a plurality of combined point clouds.

Clause 8. The dental scanning system of clause 7, further comprising: registering each of the combined point clouds to generate a 3D model of an external surface of the patient's face.

Clause 9. The dental scanning system of clause 8, wherein generating a 3D model of the patient's face and teeth using the 2D image data, the extraoral 3D image data, and the intraoral 3D image data includes combining the a 3D model of an external surface of the patient's face with the 2D image data and the intraoral 3D image data.

Clause 10. The dental scanning system of clause 1, further comprising a flash, wherein capturing 2D image data of a patient's face with the extraoral imaging device includes capturing the 2D image when the flash activates and a light from the flash illuminates the face the patient with at least twice an ambient illumination.

Clause 11. The dental scanning system of clause 10, wherein illumination on the face of the patient from the flash is at least 1400 lux.

Clause 12. The dental scanning system of clause 10, wherein the flash includes at least two light emitters.

Clause 13. The dental scanning system of clause 1, wherein the 3D imaging cameras are IR imaging cameras.

Clause 14. The dental scanning system of clause 12, further comprising one or more IR emitting structured light projectors.

Clause 15. The dental scanning system of clause 1, wherein capturing extraoral 3D image data of the patient's face includes capturing at least two stereoscopic imaging pairs with the extraoral imaging device of each of a left, right, and front of the patient's face.

Clause 16. The dental scanning system of clause 1, wherein capturing extraoral 3D image data of the patient's face includes repeatedly capturing at least two stereoscopic imaging pairs as the patient moves their head from one side to the other side.

Clause 17. The dental scanning system of clause 1, wherein the method further comprises determining a final position of a patient's teeth after dental treatment and generating a model of the final position of a patient's teeth after dental treatment based on the intraoral 3D data.

Clause 18. The dental scanning system of clause 17, wherein the method further comprises: capturing 2D images of the patient's face using the extraoral imaging device; combining the model of the final position of a patient's teeth after dental treatment with the 2D images to generate a facial image of the patient after treatment; and displaying the facial image of the patient after treatment within 250 ms of capturing the 2D images of the patient's face.

Clause 19. The dental scanning system of clause 1, wherein capturing 2D image data of a patient's face with the extraoral imaging device occurs simultaneously with capturing extraoral 3D image data of the patient's face including at least two stereoscopic imaging pairs with the extraoral imaging device.

Clause 20. The dental scanning system of clause 1, wherein capturing 2D image data of a patient's face with the extraoral imaging device occurs within 500 ms of capturing extraoral 3D image data of the patient's face including at least two stereoscopic imaging pairs with the extraoral imaging device.

Clause 21. The dental scanning system of clause 1, wherein the method further comprises modeling dynamic occlusion of the patient's upper and lower arches.

Clause 22. The dental scanning system of clause 21, wherein modeling the dynamic occlusion of the patient's upper and lower arches includes modeling the relative positions of the patient's upper and lower jaws.

Clause 23. The dental scanning system of clause 22, wherein modeling the relative positions of the patient's upper and lower jaws includes capturing dynamic occlusion images with the extraoral imaging device as the patient moves their jaws during occlusion.

Clause 24. The dental scanning system of clause 23, wherein modeling the relative positions of the patient's upper and lower jaws includes determining a position of a digital model of the patient's lower arch relative to a digital model of the patient's upper arch based on the dynamic occlusion images.

Clause 25. The dental scanning system of clause 1, wherein the extraoral imaging device includes a secondary imaging device that is couplable to the extraoral imaging device and comprise a second image capturing device.

Clause 26. The dental scanning system of clause 25, wherein the method includes capturing 2D images of the patient's upper and lower arch from an occlusal perspective.

Clause 27. A dental scanning method, the method comprising: providing directions to capture an intraoral scan data of a dentition of a patient using an intraoral scanner; receiving intraoral scan data of the dentition of the patient from the intraoral scanner; automatically formatting the intraoral scan data for a patient data file; automatically associating the intraoral scan data with the patient data file; providing directions to capture a 3D face scan of a face of the patient with an extraoral imaging device; receiving 3D extraoral image data including a 3D face scan of the face of the patient from the extraoral imaging device; automatically formatting the 3D extraoral image data for the patient data file; and automatically associating the 3D extraoral image data with the patient data file.

Clause 28. The dental scanning method of clause 27, further comprising: providing directions to capture 2D extraoral image data of the dentition of the patient with the extraoral imaging device; receiving 2D extraoral image data of the dentition of the patient from the extraoral imaging device; formatting the 2D extraoral image data for the patient data file; and associating the 2D extraoral image data with the patient data file.

Clause 29. The dental scanning method of clause 28, further comprising: providing directions to capture 2D image data of the face of the patient using the extraoral imaging device; receiving 2D image data of the face of the patient from the extraoral imaging device; formatting the 2D image data of the face of the patient for the patient data file; and associating the 2D image data of the face of the patient with the patient data file.

Clause 30. A non-transitory computer readable medium having instructions stored thereon that when executed by a processor cause the processor to carry out the method of any one of clauses 27-29.

Clause 31. A dental scanning system, the system comprising: an extraoral imaging device including a color imaging camera and three 3D imaging cameras configured to capture at least two stereoscopic image pairs; and a control unit having a processor and non-transitory computer readable medium having instructions stored thereon that when executed by the processor cause the system to carry out the method of any one of clauses 27-29 and to carry out the steps of: generating a 3D model of the patient's face and teeth using the 2D image data, the extraoral 3D image data, and the intraoral 3D image data.

Clause 32. The dental scanning system of clause 31, further comprising: a control unit including the processor and the non-transitory computer readable medium, and wherein the intraoral scanner and the extraoral imaging device are coupled to the control unit.

Clause 33. The dental scanning system of clause 32, further comprising: a portable cart, wherein the control unit, the extraoral imaging device, and the intraoral scanner are coupled to the portable cart.

Clause 34. The dental scanning system of clause 33, further comprising: a display coupled to the cart and the control unit.

Clause 35. The dental scanning system of clause 31, wherein capturing the capturing extraoral 3D image data of the patient's face including one or more stereoscopic imaging pairs with the extraoral imaging device includes capturing the least two stereoscopic imaging within 30 ms of each other.

Clause 36. The dental scanning system of clause 31, wherein capturing the capturing extraoral 3D image data of the patient's face including one or more stereoscopic imaging pairs with the extraoral imaging device includes capturing the least two stereoscopic imaging simultaneously.

Clause 37. The dental scanning system of clause 31, further comprising: generating a pair of point clouds from each stereoscopic imaging pair; and combining each respective pair of point clouds to generate a plurality of combined point clouds.

Clause 38. The dental scanning system of clause 34, further comprising: registering each of the combined point clouds to generate a 3D model of an external surface of the patient's face.

Clause 39. The dental scanning system of clause 35, wherein generating a 3D model of the patient's face and teeth using the 2D image data, the extraoral 3D image data, and the intraoral 3D image data includes combining the a 3D model of an external surface of the patient's face with the 2D image data and the intraoral 3D image data.

Clause 40. The dental scanning system of clause 31, further comprising a flash, wherein capturing 2D image data of a patient's face with the extraoral imaging device includes capturing the 2D image when the flash activates and a light from the flash illuminates the face the patient with at least twice an ambient illumination.

Clause 41. The dental scanning system of clause 40, wherein illumination on the face of the patient from the flash is at least 1400 lux.

Clause 42. The dental scanning system of clause 40, wherein the flash includes at least two light emitters.

Clause 43. The dental scanning system of clause 31, wherein the 3D imaging cameras are IR imaging cameras.

Clause 44. The dental scanning system of clause 36, further comprising one or more IR emitting structured light projectors.

Clause 45. The dental scanning system of clause 31, wherein capturing extraoral 3D image data of the patient's face includes capturing at least two stereoscopic imaging pairs with the extraoral imaging device of each of a left, right, and front of the patient's face.

Clause 46. The dental scanning system of clause 31, wherein capturing extraoral 3D image data of the patient's face includes repeatedly capturing at least two stereoscopic imaging pairs as the patient moves their head from one side to the other side.

Clause 47. The dental scanning system of clause 31, wherein the method further comprises determining a final position of a patient's teeth after dental treatment and generating a model of the final position of a patient's teeth after dental treatment based on the intraoral 3D data.

Clause 48. The dental scanning system of clause 31, wherein the method further comprises: capturing 2D images of the patient's face using the extraoral imaging device; combining the model of the final position of a patient's teeth after dental treatment with the 2D images to generate a facial image of the patient after treatment; and displaying the facial image of the patient after treatment within 250 ms of capturing the 2D images of the patient's face.

Clause 49. The dental scanning system of clause 31, wherein capturing 2D image data of a patient's face with the extraoral imaging device occurs simultaneously with capturing extraoral 3D image data of the patient's face including at least two stereoscopic imaging pairs with the extraoral imaging device.

Clause 50. The dental scanning system of clause 31, wherein capturing 2D image data of a patient's face with the extraoral imaging device occurs within 500 ms of capturing extraoral 3D image data of the patient's face including at least two stereoscopic imaging pairs with the extraoral imaging device.

Clause 51. The dental scanning system of clause 31, wherein the method further comprises modeling dynamic occlusion of the patient's upper and lower arches.

Clause 52. The dental scanning system of clause 51, wherein modeling the dynamic occlusion of the patient's upper and lower arches includes modeling the relative positions of the patient's upper and lower jaws.

Clause 53. The dental scanning system of clause 52, wherein modeling the relative positions of the patient's upper and lower jaws includes capturing dynamic occlusion images with the extraoral imaging device as the patient moves their jaws during occlusion.

Clause 54. The dental scanning system of clause 53, wherein modeling the relative positions of the patient's upper and lower jaws includes determining a position of a digital model of the patient's lower arch relative to a digital model of the patient's upper arch based on the dynamic occlusion images.

Clause 55. The dental scanning system of clause 31, wherein the extraoral imaging device includes a secondary imaging device that is couplable to the extraoral imaging device and comprise a second image capturing device.

Clause 56. The dental scanning system of clause 55, wherein the method includes capturing 2D images of the patient's upper and lower arch from an occlusal perspective.

Clause 57. A dental scanning system, the system comprising: an intraoral scanner; an extraoral imaging device including a color imaging camera couplable in electronic communication with the intraoral scanner; and non-transitory computer readable medium with instructions that, when executed by a processor, cause the system to carry out a method, the method including: capturing 2D image data of a patient's face and teeth with the extraoral imaging device; capturing intraoral 3D image data of a dentition of the patient with the intraoral scanner; generating a 3D model of the patient's teeth based on the intraoral 3D image data; and associating the 2D image data of the patient's face with the 3D model.

Clause 58. The dental scanning system of clause 57, further comprising: generating a treatment plan based on the associated 2D image data and the 3D model.

Clause 59. The dental scanning system of clause 57, wherein the intraoral scanner includes the extraoral imaging device at a proximal end of the intraoral scanner and a 3D imaging device at a distal end.

Clause 60. The dental scanning system of clause 57, further comprising a sleeve configured to be received over a distal end of the intraoral scanner, the sleeve including the extraoral imaging device.

Clause 61. The dental scanning system of clause 60, wherein the sleeve includes an internal cavity sized and shaped to receive the distal end of the intraoral scanner therein.

Clause 62. The dental scanning system of clause 60, wherein the sleeve includes the color imaging camera, the color imaging camera comprising a lens and image sensor configured to capture 2D images.

Clause 63. The dental scanning system of clause 62, further comprising a polarizing filter configured to polarize light captured by the image sensor.

Clause 64. The dental scanning system of clause 63, wherein the polarizing filter is located in front of the lens, between the lens and the patient when in use.

Clause 65. The dental scanning system of clause 63, wherein the polarizing filter is located between the lens and the sensor.

Clause 66. The dental scanning system of clause 62, wherein the sleeve includes a light source configured to illuminate a field of view of the color imaging camera.

Clause 67. The dental scanning system of clause 66, further comprising a polarizing filter configured to polarize the light from the light source.

Clause 68. The dental scanning system of clause 67, wherein the polarizing filter is configured to be between the light source and the patient when in use.

Clause 69. The dental scanning system of clause 62, wherein the sleeve further comprises a heating element.

Clause 70. The dental scanning system of clause 69, wherein the heating element is located on an opposite side of the sleeve as the imaging system.

Clause 71. The dental scanning system of clause 69, wherein the heating element is configured to be located on a side of the sleeve facing away from the patient when the patient is imaged by the imaging system.

Clause 72. The dental scanning system of clause 66, wherein the color imaging camera is coupled in electronic communication with the intraoral scanner.

Clause 73. The dental scanning system of clause 72, wherein the sleeve obscures the 3D scanner of the intraoral scanner.

Clause 74. The dental scanning system of clause 57, wherein the method further comprises: entering a 2D imaging mode to capture the 2D image data; and entering a 3D imaging mode to capture the 3D image data.

Clause 75. The dental scanning system of clause 72, wherein the method further comprises: entering the 2D imaging mode based on detecting a sleave configured to be received over a distal end of the intraoral scanner, the sleave including the extraoral imaging device.

Clause 76. The dental scanning system of clause 57, wherein the method further comprises: entering a disease screening mode.

Clause 77. The dental scanning system of clause 76, further comprising: a disease screening system couplable to the intraoral scanner.

Clause 78. The dental scanning system of clause 77, wherein the disease screening system further comprises: an excitation light source; and an image sensor configured to capture images of the patient's anatomy.

Clause 79. The dental scanning system of clause 78, wherein the disease screening system further comprises: a fiber optic extending from the disease screening system to a distal end of the intraoral scanner and configured to be optically coupled to the excitation light source and the image sensor, the fiber optic positioned within a field of view of the 3D imaging system of the intraoral scanner.

Clause 80. The dental scanning system of clause 79, wherein the method further comprises: simultaneously capturing 3D image data of the patient's intraoral cavity and the fiber optic and 2D image data of the patient's intraoral cavity with the disease screening system.

Clause 81. The dental scanning system of clause 80, wherein the method further comprises: mapping the 2D image data of the patient's intraoral cavity with the disease screening system to a location in the patient's intraoral cavity based on the 3D image data.

Clause 82. The dental scanning system of clause 81, wherein the method further comprises: while in the 3D scanning mode, ignoring 3D scan data that includes moving soft tissue when generating a 3D model of the patient's teeth.

Clause 83. The dental scanning system of clause 82, wherein the method further comprises: while in the disease screening mode, using 3D scan data that includes moving soft tissue when mapping the 2D image data of the patient's intraoral cavity with the disease screening system to a location in the patient's intraoral cavity based on the 3D image data.

Clause 84. The dental scanning system of clause 83, wherein the method further comprises: displaying on the display, a real-time image of the field of view of the image sensor of the disease screening system while in the disease screening mode.

Clause 85. The dental scanning system of clause 81, wherein the method further comprises: identifying disease locations on the patient's anatomy based on the mapping.

Clause 86. The dental scanning system of clause 85, wherein the method further comprises: displaying, on the display, an intraoral cavity and the identified disease locations indicated on the intraoral cavity.

Clause 87. The dental scanning system of clause 86, wherein the displayed intraoral cavity is generated based on 3D image data generated by the intraoral scanner.

Clause 88. The dental scanning system of clause 86, wherein the displayed intraoral cavity is a generic intraoral cavity.

Clause 89. The dental scanning system of clause 72, wherein the method further comprises: displaying on a display, a real-time image of the field of view of the extraoral imaging device while in the 2D imaging mode.

Clause 90. The dental scanning system of clause 72, wherein the method further comprises: displaying on a display a plurality of desired 2D image views; and replacing a respective one of the plural of 2D image views with a respective captured 2D image having a view that corresponds to the respective 2D image view.

Clause 91. The dental scanning system of clause 72, wherein the method further comprises: displaying on a display, a real-time image of the field of view of the extraoral imaging device while in the 2D imaging mode.

What is claimed is:

1. A dental scanning system, the system comprising:
an intraoral scanner;
an extraoral imaging device including a color imaging camera and a plurality of 3D imaging cameras configured to capture at least two stereoscopic image pairs; and
non-transitory computer readable medium with instructions that, when executed by a processor, cause the system to carry out a method, the method including:
capturing 2D image data of a patient's face with the extraoral imaging device;
capturing extraoral 3D image data of the patient's face including one or more stereoscopic imaging pairs with the extraoral imaging device;
capturing intraoral 3D image data of a dentition of the patient with the intraoral scanner;
generating a 3D model of the patient's face and teeth using the 2D image data, the extraoral 3D image data, and the intraoral 3D image data.

2. The dental scanning system of claim 1, further comprising:
a control unit including the processor and the non-transitory computer readable medium, and wherein the intraoral scanner and the extraoral imaging device are coupled to the control unit.

3. The dental scanning system of claim 2, further comprising:
a portable cart, wherein the control unit, the extraoral imaging device, and the intraoral scanner are coupled to the portable cart.

4. The dental scanning system of claim 3, further comprising:
a display coupled to the cart and the control unit.

5. The dental scanning system of claim 1, wherein capturing the capturing extraoral 3D image data of the patient's face including one or more stereoscopic imaging pairs with the extraoral imaging device includes capturing the least two stereoscopic imaging simultaneously.

6. The dental scanning system of claim 1, further comprising:
generating a pair of point clouds from each stereoscopic imaging pair; and
combining each respective pair of point clouds to generate a plurality of combined point clouds.

7. The dental scanning system of claim 6, further comprising:
registering each of the combined point clouds to generate a 3D model of an external surface of the patient's face.

8. The dental scanning system of claim 7, wherein generating a 3D model of the patient's face and teeth using the 2D image data, the extraoral 3D image data, and the intraoral 3D image data includes combining the 3D model of an external surface of the patient's face with the 2D image data and the intraoral 3D image data.

9. The dental scanning system of claim 1, further comprising a flash, wherein capturing 2D image data of a patient's face with the extraoral imaging device includes capturing the 2D image when the flash activates and a light from the flash illuminates the face the patient with at least twice an ambient illumination.

10. The dental scanning system of claim 9, wherein illumination on the face of the patient from the flash is at least 1400 lux.

11. The dental scanning system of claim 1, wherein the 3D imaging cameras are IR imaging cameras.

12. The dental scanning system of claim 1, further comprising one or more IR emitting structured light projectors.

13. The dental scanning system of claim 1, wherein capturing extraoral 3D image data of the patient's face includes capturing at least two stereoscopic imaging pairs with the extraoral imaging device of each of a left, right, and front of the patient's face.

14. A dental scanning system, the system comprising:
an intraoral scanner;
an extraoral imaging device including a color imaging camera couplable in electronic communication with the intraoral scanner; and
non-transitory computer readable medium with instructions that, when executed by a processor, cause the system to carry out a method, the method including:
capturing 2D image data of a patient's face and teeth with the extraoral imaging device;
capturing intraoral 3D image data of a dentition of the patient with the intraoral scanner;
generating a 3D model of the patient's teeth based on the intraoral 3D image data; and
associating the 2D image data of the patient's face with the 3D model.

15. The dental scanning system of claim 14, further comprising a sleeve configured to be received over a distal end of the intraoral scanner, the sleeve including the extraoral imaging device.

16. The dental scanning system of claim 15, wherein the sleeve includes an internal cavity sized and shaped to receive the distal end of the intraoral scanner therein.

17. The dental scanning system of claim 15, wherein the sleeve includes the color imaging camera, the color imaging camera comprising a lens and image sensor configured to capture 2D images.

18. The dental scanning system of claim 17, wherein the sleeve includes a light source configured to illuminate a field of view of the color imaging camera.

19. The dental scanning system of claim 18, further comprising a polarizing filter configured to polarize the light from the light source.

20. The dental scanning system of claim 19, wherein the polarizing filter is configured to be between the light source and the patient when in use.
